(19) United States
(12) Patent Application Publication
(10) Pub. No.: US 2024/0327866 A1
(43) Pub. Date: Oct. 3, 2024

(54) REGULATED VIRAL DELIVERY SYSTEMS AND THEIR USES

(71) Applicant: The Regents of the University of California, Oakland, CA (US)

(72) Inventors: Noriyuki Kasahara, Oakland, CA (US); Sara A. Collins, Oakland, CA (US); Alexander F. Haddad, Oakland, CA (US); Manish Aghi, Oakland, CA (US); Christopher Logg, Oakland, CA (US)

(73) Assignee: The Regents of the University of California, Oakland, CA (US)

(21) Appl. No.: 18/579,829

(22) PCT Filed: Jul. 21, 2022
(86) PCT No.: PCT/US2022/074024

§ 371 Date: Jan. 16, 2024

Related U.S. Application Data
(60) Provisional application No. 63/224,330, filed on Jul. 21, 2021.

(51) Int. Cl. C12N 15/86; A61P 35/00

(52) U.S. Cl. CPC C12N 15/86; A61P 35/00; C12N 2740/13043; C12N 2800/40; C12N 2820/002; C12N 2830/001

(57) ABSTRACT

Provided herein are recombinant virus systems comprising two or more coordinatedly regulated viral vectors. Methods for treating a cell proliferative disorder using these systems are also provided. The compositions and methods provided herein provide more efficient and coordinated delivery of larger transgenes and/or larger quantities of a tansgene than previous methods by avoiding receptor interference and/or superinfection resistance, thereby enabling progressive replication of both vectors and efficient gene delivery by each vector.

### PRIOR RELATED APPLICATIONS

This application claims the benefit of U.S. Provisional Application No. 63/224,330 filed on Jul. 21, 2021, which is hereby incorporated by reference in its entirety.

### BACKGROUND

Although progress has been made in viral gene therapy, it is often difficult to deliver large amounts of genetic information that are stably and persistently expressed throughout a solid tumor mass. Existing technologies often utilize a single non-replicating or replicating viral vector to deliver transgenes. Even if two or more vectors are combined, they are not coordinately regulated in a manner that enables synchronized replication as well as transgene expression. Therefore, compositions and methods for efficient and coordinated delivery of larger transgenes and/or larger quantities of a transgene are necessary.

### SUMMARY

Provided herein are recombinant viral systems comprising two or more viral vectors, which are coordinately regulated. In some embodiments, the recombinant viral system is a recombinant retrovirus system comprising two or more retroviral vectors, which are coordinately regulated. The inventors have discovered that coordinately regulating viral vectors, for example, retroviral vectors, avoids receptor interference and/or superinfection resistance, thereby enabling progressive replication of both vectors and efficient gene delivery by each vector.

Provided herein is a recombinant viral system comprising: (a) a first virus (i) encoding a first regulatory element operably linked to a nucleic acid encoding a first activator; and (ii) lacking a coding sequence for at least one viral protein required for replication such that the first virus is a replication-deficient virus; and (b) a second virus comprising a nucleic acid comprising a first polynucleotide encoding the viral protein or proteins necessary for viral replication that are lacking in the first virus, wherein the first polynucleotide is only expressed when the first activator activates expression of the first polynucleotide and/or its encoded viral protein(s).

In some embodiments, the recombinant viral system is a recombinant retrovirus system. Therefore, provided herein is a recombinant retrovirus system comprising: (a) a first retrovirus (i) encoding a first regulatory element operably linked to a nucleic acid encoding a first activator; and (ii) lacking a coding sequence for at least one viral protein required for replication such that the first retrovirus is a replication-deficient retrovirus (RDV); and (b) a second retrovirus comprising a nucleic acid comprising a first polynucleotide encoding the viral protein or proteins necessary for viral replication that are lacking in the first retrovirus, wherein the first polynucleotide is only expressed when the first activator activates expression of the first polynucleotide and/or its encoded viral protein(s).

In some embodiments, the activator activates expression by increasing transcription or translation of the first polynucleotide. In some embodiments, the second virus, for example, a retrovirus, comprises a second regulatory element operably linked to the first polynucleotide encoding a viral protein necessary for viral replication, and wherein the first activator activates transcription of the first polynucleotide by binding to the second regulatory element. In some embodiments, the activator binds to the second regulatory element to activate transcription, wherein the second regulatory element is a promoter, an enhancer or a repressor binding sequence.

In some embodiments, the first activator is a de-repressor; and the first polynucleotide sequence encoded by the second virus, for example, a retrovirus, is only expressed when the de-repressor activates expression by dc-repressing expression of the first polynucleotide and/or viral protein(s). In some embodiments, de-repression occurs at the transcriptional level or the translational level.

In some embodiments, the first regulatory element and/or the second regulatory element is selected from the group consisting of a promoter, an enhancer, a promoter/enhancer combination, an internal ribosome entry site, an epigenetic regulator and a translation regulator. In some embodiments, the promoter is a constitutive or an inducible promoter. In some embodiments, the second virus is a replicating virus, for example, a replicating retrovirus (RRV) encoding all viral proteins necessary for viral replication. In some embodiments, the first and/or second virus, for example, a retrovirus, further comprises a heterologous expression cassette comprising a payload promoter operably linked to a payload polynucleotide sequence.

In some embodiments, the viral protein required for replication is selected from the group consisting of gag, env, pol, rev and tat. In some embodiments, the retroviruses are selected from the group consisting of lentivirus, murine leukemia virus (MLV). Moloney murine leukemia virus (MoMLV), foamy virus. In some embodiments, the payload polynucleotide encodes a polypeptide selected from the group consisting of a therapeutic protein, a prodrug activator, a cytotoxic protein, and a reporter protein. In some embodiments, the prodrug activator is thymidine kinase, cytidine deaminase or purine nucleoside phosphorylase (PNP).

In some embodiments, the first activator is selected from the group consisting of HIV-1 trans-activator protein (Tat), HIV-1 Rev, Gal4-VP16, GAL4FF, GAL4-VP64, and VP16-E2 and tetracycline transactivator protein.

In some embodiments, the second virus is a replication-deficient virus, for example, replication-deficient retrovirus (RDV) and encodes a third regulatory element operably linked to a nucleic acid encoding a second activator; and the first virus, for example, a retrovirus comprises a nucleic acid comprising a second polynucleotide encoding a viral protein necessary for viral replication, wherein the second polynucleotide is only expressed when the second activator activates expression of the second polynucleotide, and wherein the first and second virus, for example, retroviruses, can only replicate when the first and second activators are expressed.

In some embodiments, the first virus, for example, a retrovirus, comprises a fourth regulatory element operably linked to the second polynucleotide encoding a viral protein necessary for viral replication, and wherein the first activator activates transcription of the second polynucleotide by binding to the fourth regulatory element. In some embodiments, the third regulatory element or the fourth regulatory element is selected from the group consisting of a promoter, an enhancer, a promoter/enhancer combination, an internal ribosome entry site, an epigenetic regulator and a translational regulator. In some embodiments, the promoter is a constitutive or an inducible promoter.

In some embodiments, the first or second virus (e.g., the first retrovirus or second retrovirus), or both, comprise a heterologous expression cassette comprising a payload promoter operably linked to a payload polynucleotide. In some embodiments, the payload polynucleotide encodes a polypeptide selected from the group consisting of a therapeutic protein, a prodrug activator, a cytotoxic protein, and a reporter protein.

In some embodiments, the viral protein required for replication is selected from the group consisting of gag, env, pol, rev and tat. In some embodiments, the retroviruses are selected from the group consisting of lentivirus, murine leukemia virus (MLV). Moloney murine leukemia virus (MoMLV), and foamy virus.

In some embodiments, the payload polynucleotide encodes a polypeptide selected from the group consisting of a therapeutic protein, a prodrug activator, a cytotoxic protein, and a reporter protein. In some embodiments, the prodrug activator is thymidine kinase, cytidine deaminase or purine nucleoside phosphorylase (PNP).

In some embodiments, the third or fourth regulatory element is selected from the group consisting of a constitutive promoter, an inducible promoter, and a tissue-specific promoter. In some embodiments, the activator is selected from the group consisting of HIV-1 trans-activator protein (Tat), HIV-1 Rev, Gal4-VP16, and VP16-E2 and tetracycline transactivator protein.

In some embodiments, the vectors described herein comprise one or more long terminal repeat (LTR) sequences. Any of the LTR sequences, for example, a 3′LTR and/or a 5′LTR, in any of the vectors described herein, can comprise a deletion, for example, a deletion in the U3 region of the LTR. In some embodiments, the 3′LTR and/or the 5′ LTR comprise a heterologous promoter (e.g., a CMV promoter). In some embodiments, the 3′ LTR sequence is a retroviral 3′LTR sequence (e.g., a MMLV 3′LTR sequence) that has been modified to reduce or disrupt native promoter function in the 3′LTR. Native promoter function can be reduced or disrupted, for example, by deleting one or more sequences in the 3′LTR and/or inserting one or more sequences. In some embodiments, one or more nucleic acid sequences comprising a binding site for an activator, for example, one, two, three, four, five, six, seven, eight, nine, ten or more GAL4 binding sites, are inserted into the LTR. In some embodiments, the 3′LTR sequence of any vector described herein comprises a nucleic acid sequence having at least 60%, 70%, 80%/u, 90%, 95%, or 99% identity to SEQ ID NO: 1, SEQ ID NO: 2 or SEQ ID NO: 3.

Provided herein is a method for making a recombinant virus system, for example, a retrovirus system, comprising: (a) transfecting a first suitable host cell with the first viral vector of any of the systems described herein; (b) transfecting a second suitable host cell with the second viral vector of any of the systems provided herein; and (c) recovering the first and second viruses.

Provided herein is a method for transducing a target cell with a replicating retrovirus system comprising contacting the target cell with the first virus and second virus of any one of the systems described herein.

In some embodiments, the cell is a mammalian cell. In some embodiments, the cell is contacted in vitro, ex vivo or in vivo.

Also provided is a pharmaceutical composition comprising: (a) the first virus and/or the second virus of any of the systems provided herein; (b) and a pharmaceutical carrier.

Also provided is a method of treating a disease in a subject in need thereof comprising administering any of the systems or pharmaceutical compositions provided herein to the subject. In some methods, the disease is a cell proliferative disorder. In some embodiments, the cell proliferative disorder is selected from the group consisting of lung cancer, breast cancer, ovarian cancer, uterine cancer, prostate cancer, testicular cancer, kidney cancer, urinary tract cancer, oral cancer, head and neck cancer, esophageal cancer, gastric cancer, pancreatic cancer, colorectal cancer, skin cancer, melanoma, sarcoma, lymphoma, leukemia, and brain cancer including glioblastoma, anaplastic astrocytoma, oligodendroglioma, medulloblastoma. In some embodiments, the cancer is glioblastoma.

In some embodiments, the first or second polynucleotide encodes a prodrug activator, and wherein the method further comprises administering a prodrug to the subject, such that when the prodrug activator is expressed, the prodrug activator converts the prodrug into a toxic drug. In some embodiments, the first viral vector and/or second viral vector, for example, a first retroviral vector and/or a second retroviral vector is administered to the subject as a plasmid or as an infectious viral particle. In some embodiments, the subject is a mammal. In some embodiments, the subject is a human.

In some embodiments, administration is systemic, topical or local administration. In some embodiments, the first and second retrovirus of the system are administered simultaneously or sequentially to the subject.

### BRIEF DESCRIPTION OF THE FIGURES

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 is a schematic of the plXIX GAL4 IRES EMD retroviral vector, an exemplary replication deficient retrovirus (RDV) comprising a GAL4/VP16 activator (GAL4FF), as described in the Examples.

FIG. 2 is a schematic of the pAC3_P2A_Stb_GAL4BS_Min Deletion retroviral vector, an exemplary replicating retrovirus (RRV), described in the Examples.

FIG. 3 is a schematic of the pAC3_P2A_Stb_GAL4BS_Max Deletion retroviral vector, an exemplary replicating retrovirus (RRV), described in the Examples.

FIG. 4 is a schematic of the pLXIX Tat IRES EMD retroviral vector, an exemplary RDV, described in the Examples.

FIG. 5 is a schematic of the pAC3 TIN IRES EMD retroviral vector, an exemplary RRV, described in the Examples.

FIG. 9A is a graph showing that the pAC3_P2A_Stb_GAL4BS_Min Deletion retroviral vector demonstrates no replication in wild type SB28 tumor cells after nine days.

FIG. 9B is a graph showing that the pAC3_P2A_Stb_GAL4BS_Min Deletion replicates efficiently when GAL4/VP16 is stably expressed in SB28 tumor cells.

FIG. 10A is a graph showing that the addition of the pLXIX GAL4 IRES EMD retroviral vector and the pAC3_P2A_Stb_GAL4BS_Min Deletion retroviral vector (Minimum Strawberry vector), together, to SB28 glioblastoma tumor cells allowed for robust viral replication and spreading of both viruses.

FIG. 10B shows that co-infected cells demonstrated higher expression of Strawberry expression, highlighting the synergistic nature of the binary replication system.

FIG. 10C shows that the addition of pLXIX GAL4 IRES EMD retroviral vector and pAC3_P2A_Stb_GAL4BS_Max Deletion retroviral vector (Maximal Strawberry vector), together, to SB28 glioblastoma tumor cells allowed for robust viral replication and spreading of both viruses to a high percentage of double positive cells.

FIG. 11A is a graph showing the percentage of intracranial tumor infected by each viral construct as well as the percentage that was double infected for pAC3_P2A_Stb_GAL4BS_Min Deletion premix (Min-P) and pAC3_P2A_Stb_GAL4BS_Max Deletion premix (Max-P).

FIG. 8B is a graph showing the breakdown of viral infection subgroups as a percentage of total infected SB28 tumor cells for Min-P and Max-P.

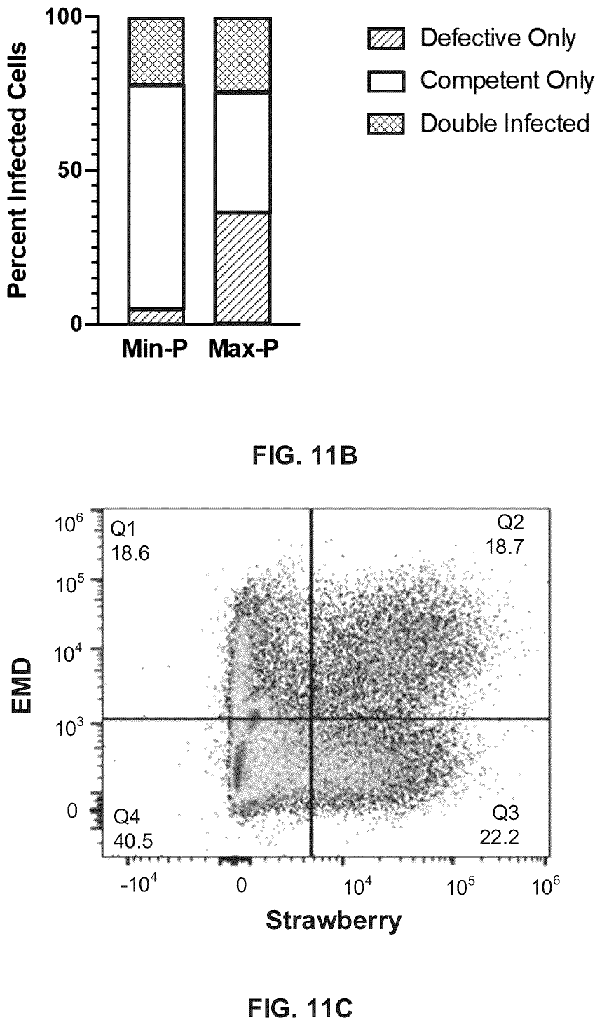

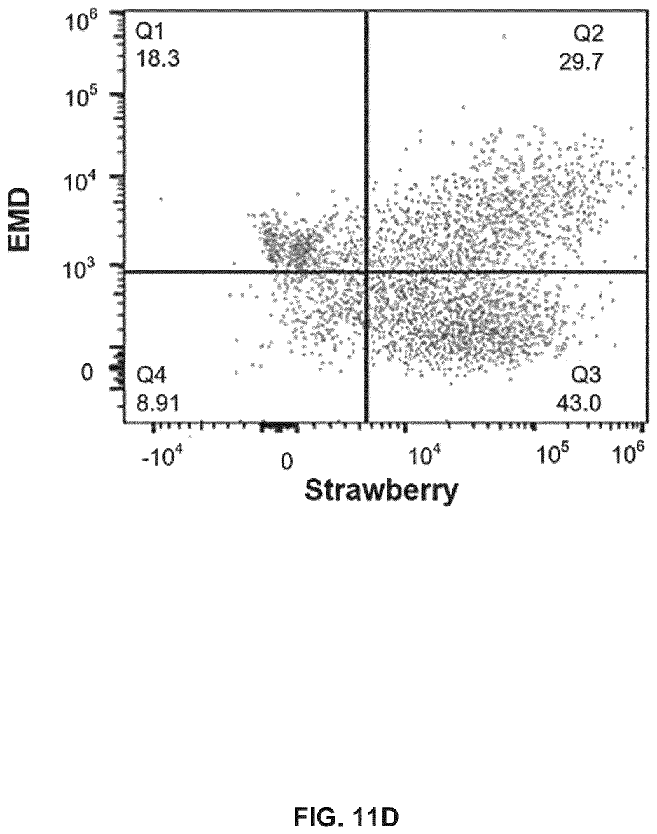

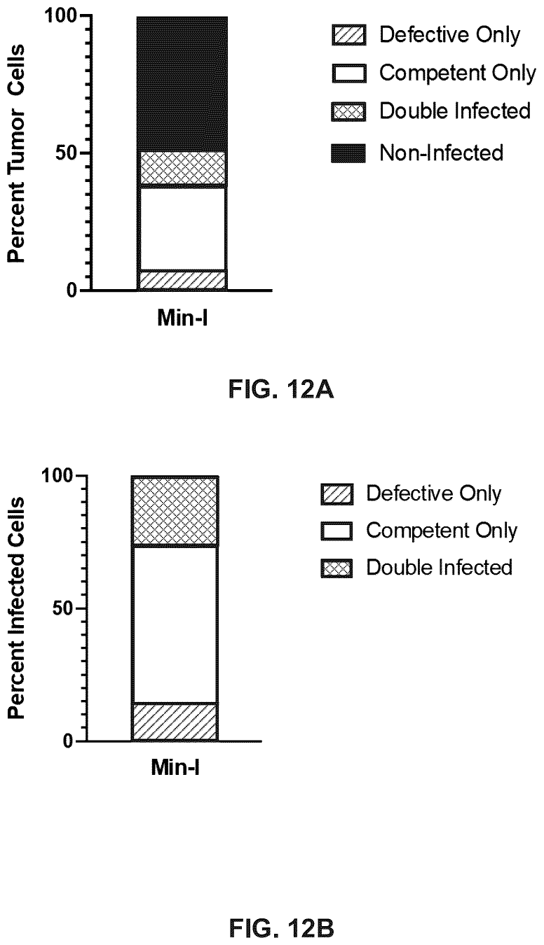

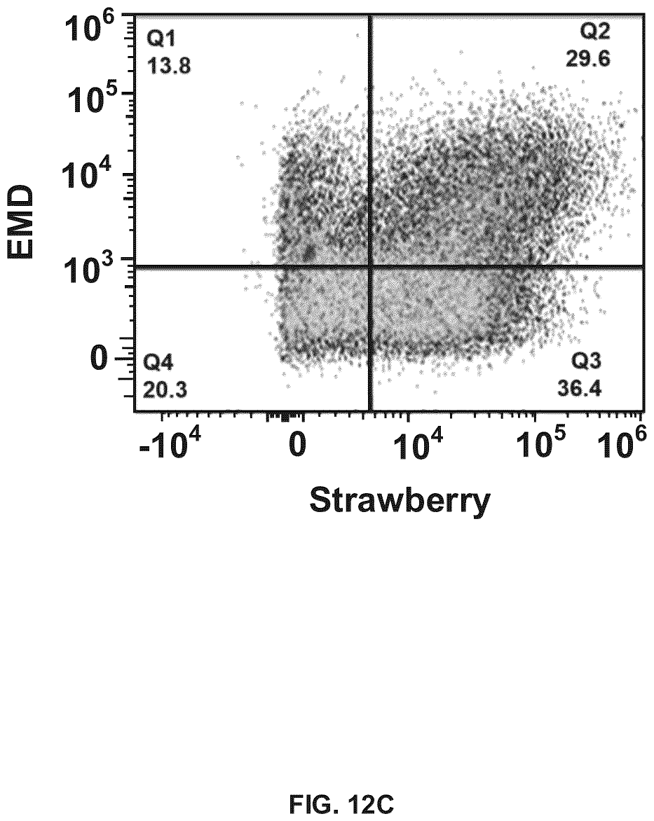

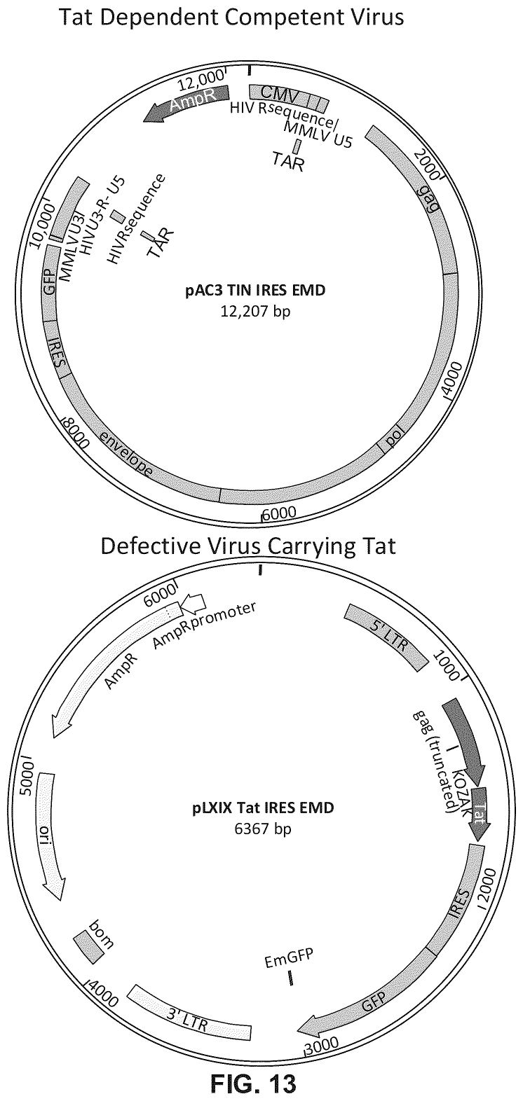

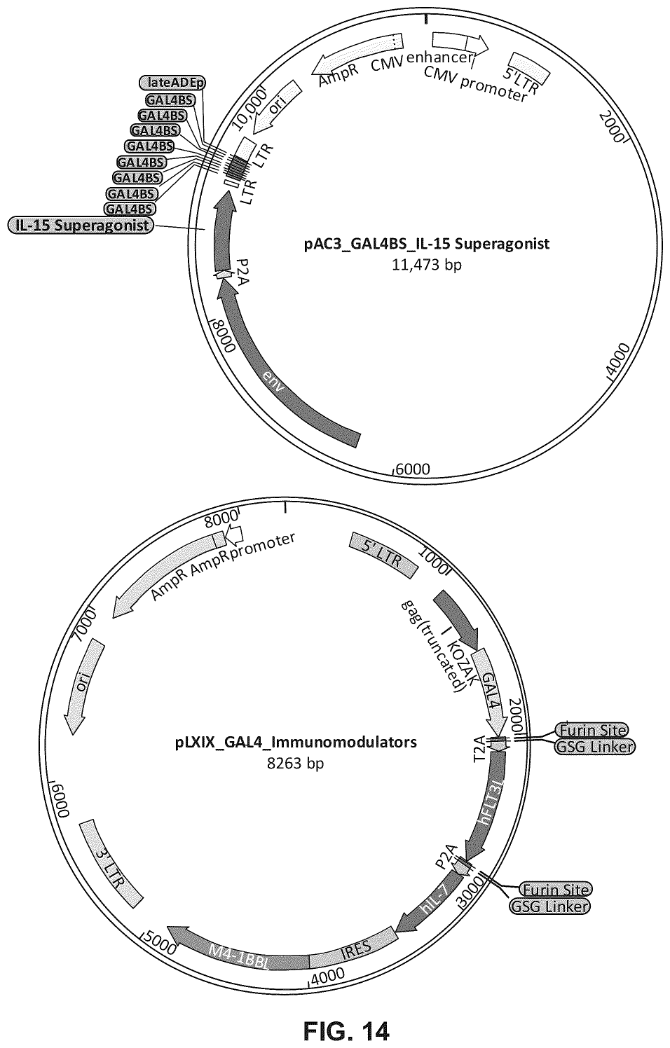

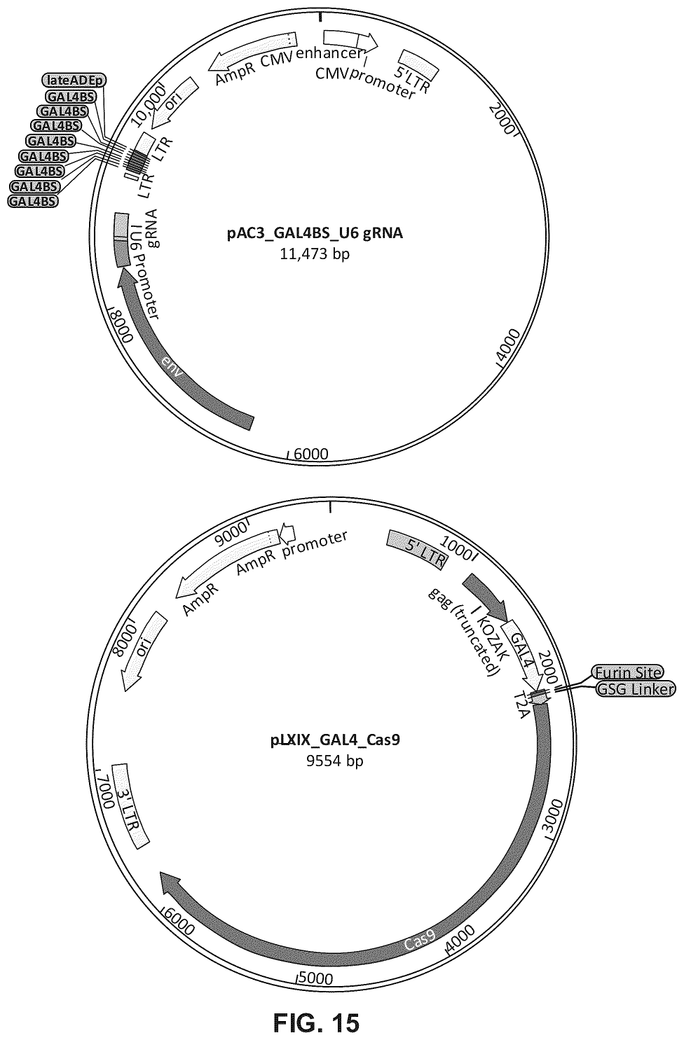

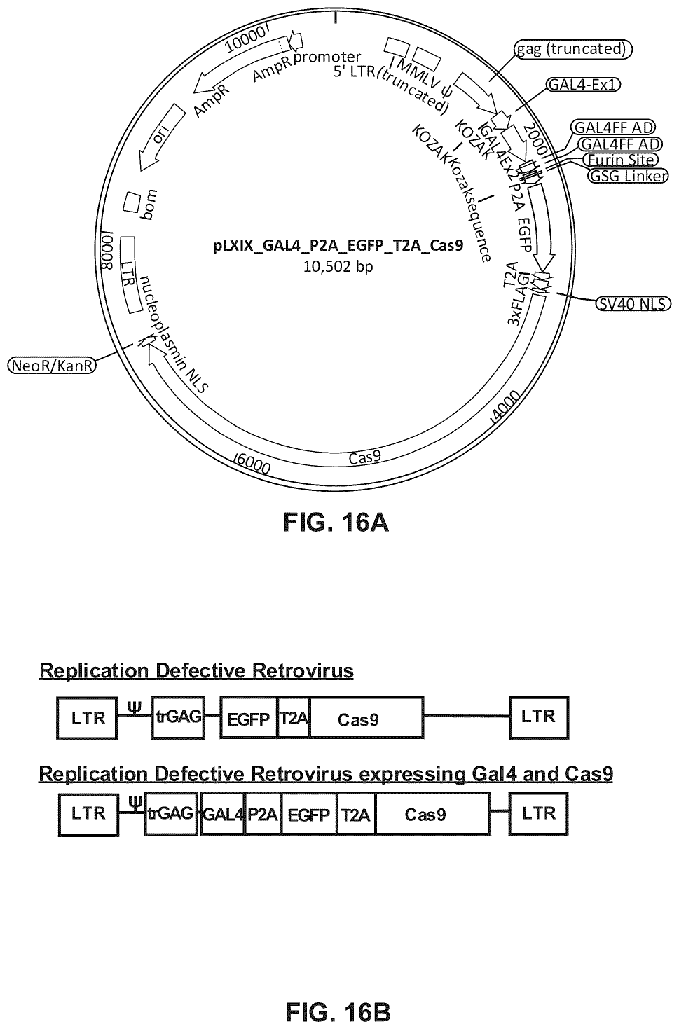

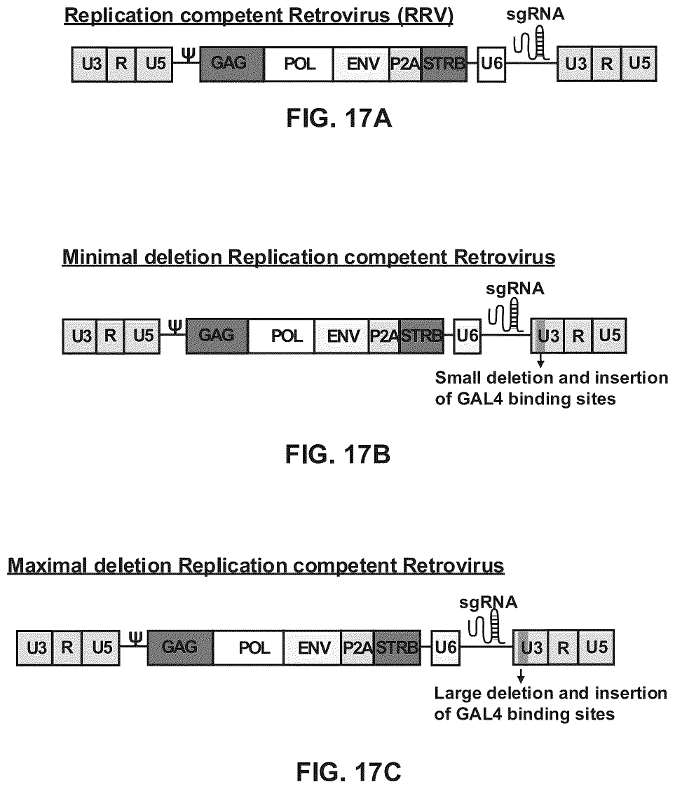

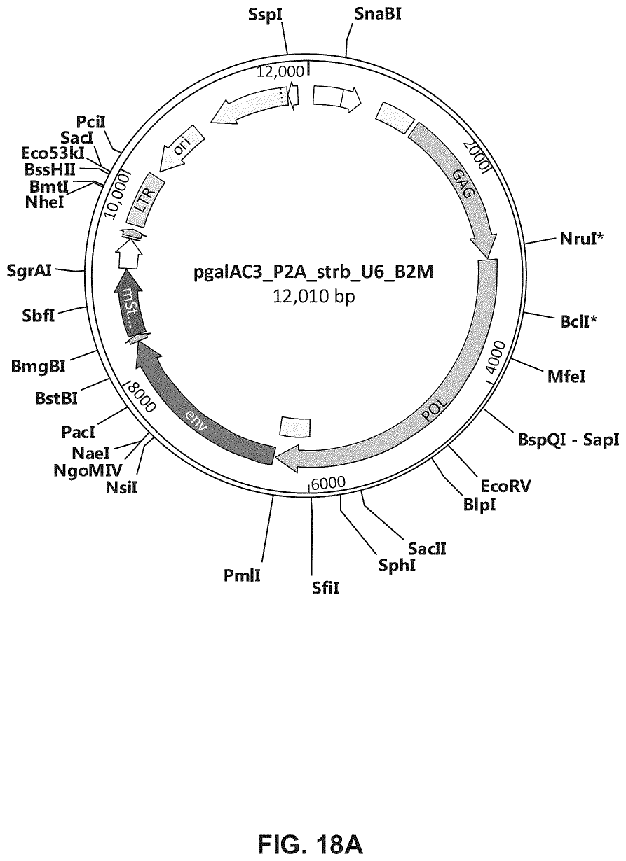

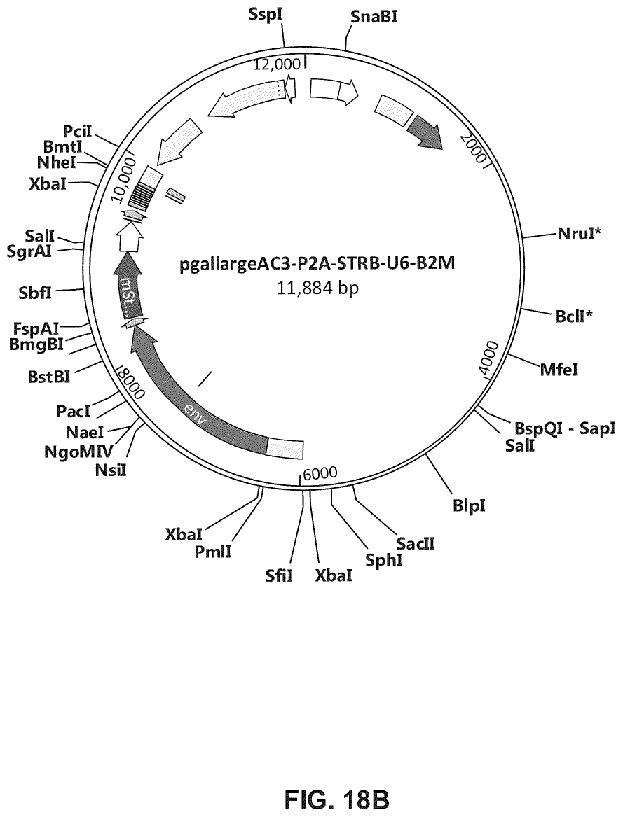

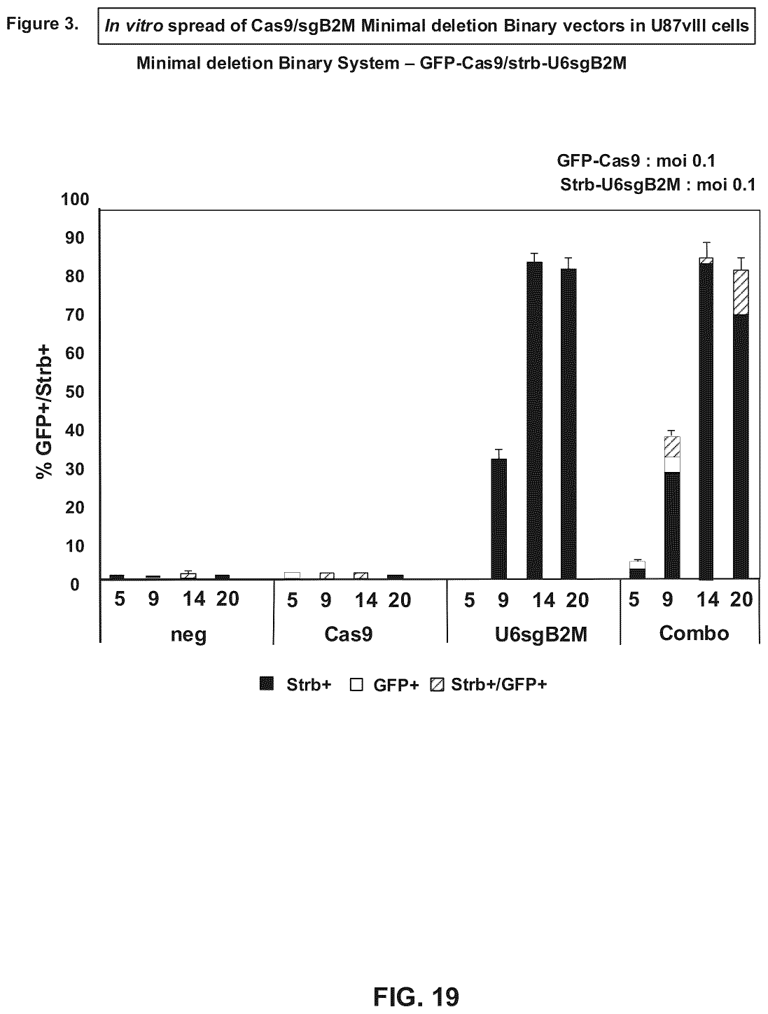

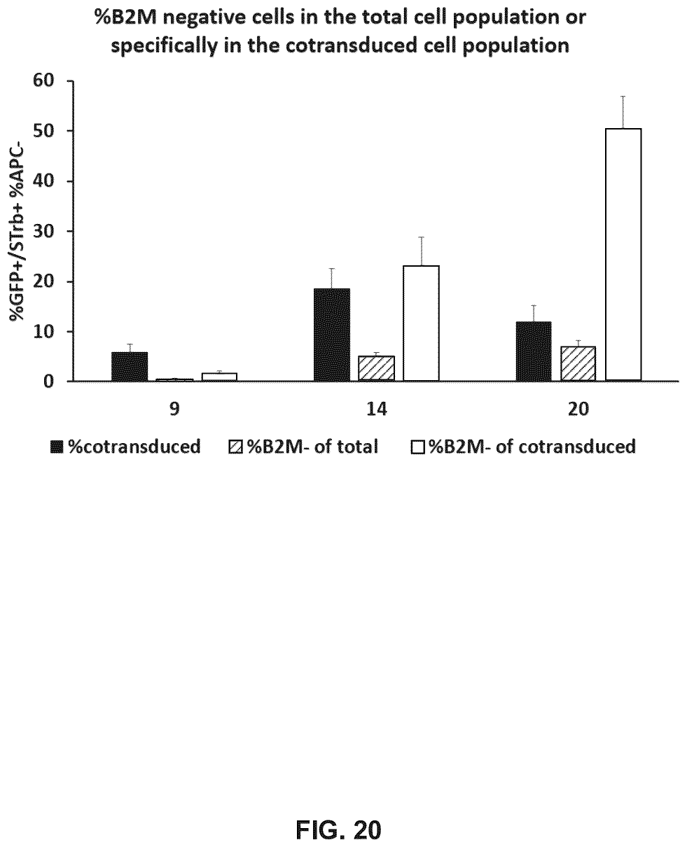

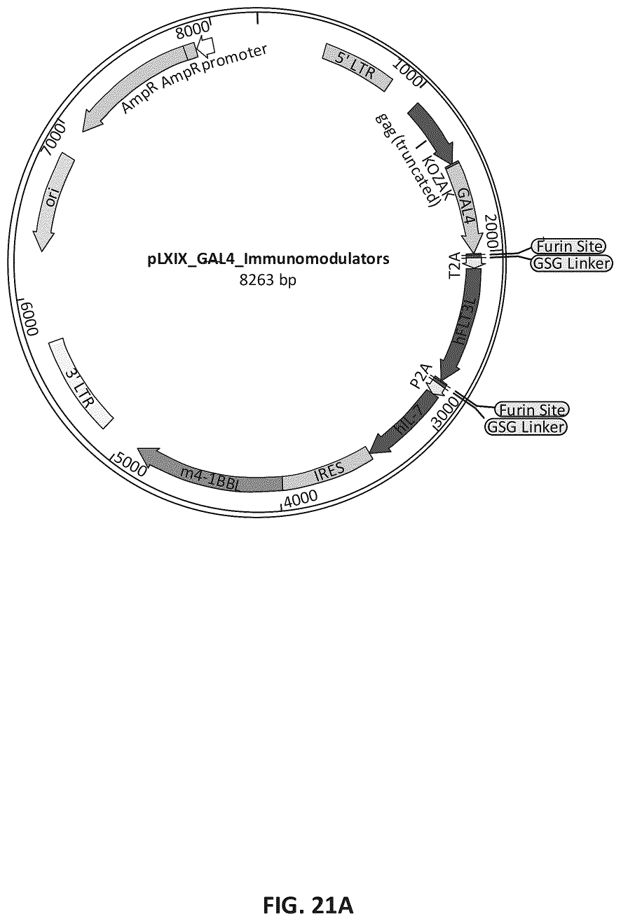

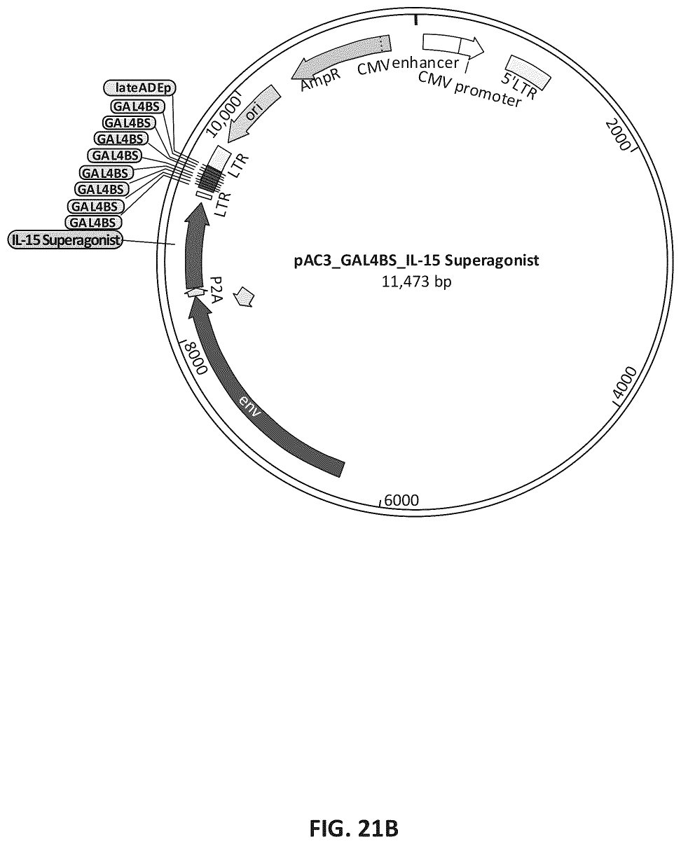

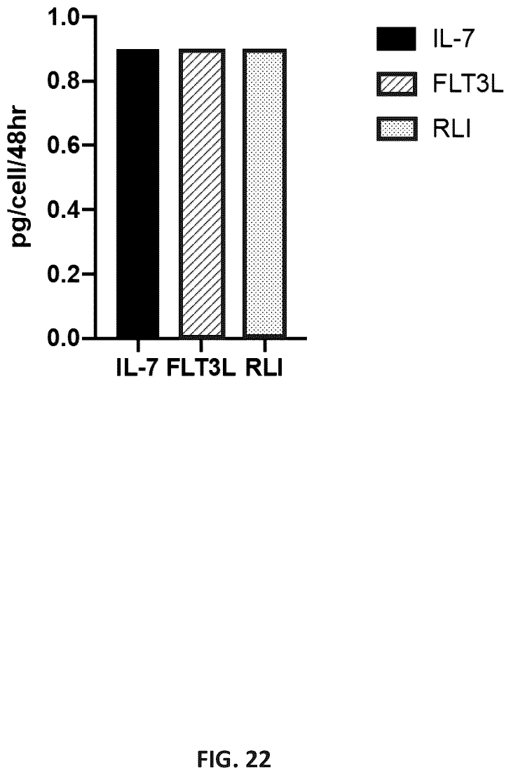

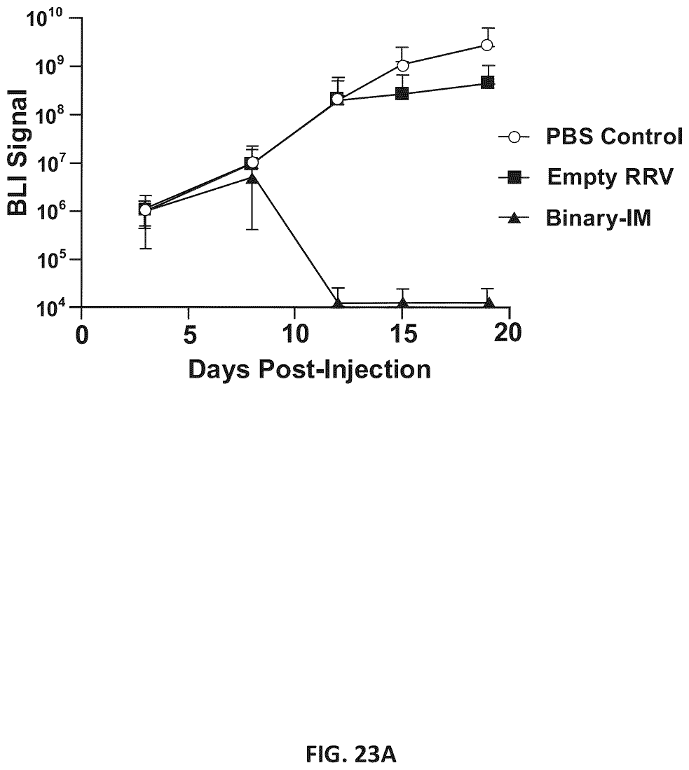

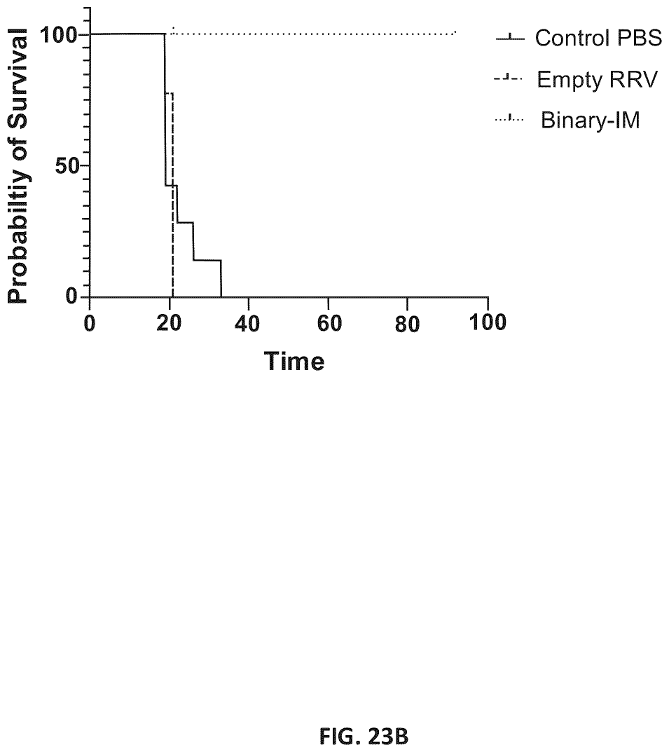

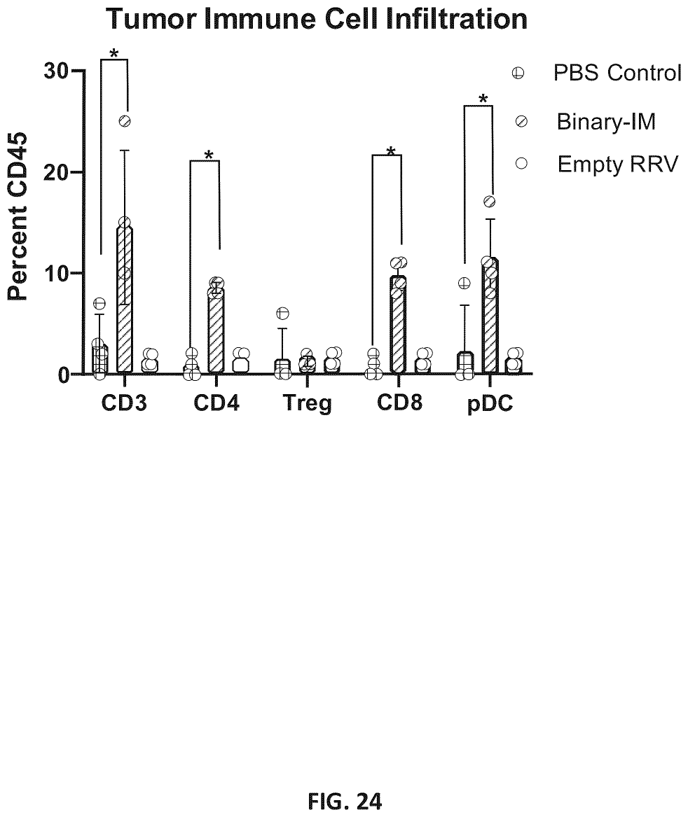

FIG. 11C is a representative flow cytometric analysis for Max-P.

FIG. 11D is a representative flow cytometric analysis for Min-P.

FIG. 12A is a graph showing the percentage of intracranial tumor infected by each viral construct as well as percent double infected for pAC3_P2A_Stb_GAL4BS_Min Deletion injection (Min-I).

FIG. 12B is a graph showing the breakdown of viral infection subgroups as a percentage of total infected SB28 tumor cells for Min-I.

FIG. 12C is a representative flow cytometric analysis for Min-I.

FIG. 13 is an exemplary retroviral vector system comprising a Tat dependent competent virus and an exemplary replication deficient retrovirus (RDV) encoding Tat.

FIG. 14 is an exemplary retroviral vector system for the delivery of an immunomodulator. In this exemplary vector, there are three immunomodulatory transcripts delivered in the defective virus. FLT3L aids with dendritic cell recruitment and development. IL-7 aids with T cell proliferation and activation. 4-1BBL is a co-stimulatory molecule that can aid with sustained T cell activation. An IL-15 superagonist (RLI) would be delivered in the coordinately regulated competent virus. This cytokine also aids with T cell activation and proliferation.

FIG. 15 is an exemplary retroviral vector system for the delivery of Cas9 to cells. This system can be used to deliver Cas9 in a defective virus. A cognate guide RNA (gRNA) for a gene of interest is delivered in the coordinately regulated competent virus. This system allows knocking out any gene for which a functional gRNA is present in tumor cells.

FIG. 16A is an exemplary replication deficient retrovirus (RDV) (pLXIX_GAL4_P2A_EGFP_T2A_Cas9) encoding Gal4 and Cas9.

FIG. 16B is a linear depiction of the expression cassette in pLXIX_GAL4_P2A_EGFP_T2A_Cas9, that encodes Gal4 and Cas9.

FIGS. 17A-C are linear depictions of exemplary replication competent retroviruses (RRV) FIG. 17A is a linear depiction of the U6-B2MsgRNA expression vector which served as a template for insertion of the U6-B2MsgRNA expression cassette into the Not1 site of the minimal (FIG. 17B) and maximal deletion RRV vectors (FIG. 17C)) using Gibson assembly.

FIG. 18A is an exemplary minimal deletion replication competent retrovirus (pgalAC3_P2A_strb_U6_B2M) encoding an sgRNA that targets β-2-microglobulin.

FIG. 18B is an exemplary maximal deletion replication competent retrovirus (pgallargeAC3_P2A_STRB_U6_B2M) encoding an sgRNA that targets β-2-microglobulin.

FIG. 19 is a graph showing the in vitro spread of replication deficient retrovirus (RDV) (pLXIX_GAL4_P2A_EGFP_T2A_Cas9) and minimal deletion replication competent retrovirus (pgalAC3_P2A_strb_U6_B2M) encoding an sgRNA that targets β-2-microglobulin, in U87vIII cells, as described in the Examples.

FIG. 20 is a graph showing confirmation of B2M knockdown in the total cell population or, specifically, in the co-transduced population using LXIX-GAL4-P2A-EGFP-T2A-Cas9 and minimal deletion AC3-P2A-Strb-U6-B2MsgRNA.

FIG. 21A is a schematic of the plXIX_GAL4_Immunomodulators retroviral vector, an exemplary replication deficient retrovirus (RDV) comprising a GAL4/VP16 activator (GAL4FF), as described in the Examples.

FIG. 21B is a schematic of the pAC3_GAL4BS_IL15 Superagonist retroviral vector described in the Examples.

FIG. 22 is a graph showing that infection of SB28, with plXIX_GAL4_Immunomodulator and pAC3-GAL4BS-IL-15, infected in a T75 plate, in 10 mL of media, efficiently secrete IL-7, RLI, and FLT3L at a concentration of 75 ng/mL or 0.09 pg/cell/48 hours.

FIG. 23A is a graph showing that treatment with pLXIX-GAL4-IM and pAC3-GAL4BS-RLI treatment leads to reduced tumor growth, as described in the Examples. Circles: PBS control; Squares: Empty RRV; Triangles: Binary-IM (administration of pLXIX-GAL4-IM and pAC3-GAL4BS-RLI).

FIG. 23B is a survival curve showing that treatment with pLXIX-GAL4-IM and pAC3-GAL4BS-RLI treatment leads to increased survival, as described in the Examples. The top line at 100% corresponds to Binary-IM (administration of pLXIX-GAL4-IM and pAC3-GAL4BS-RLI).

FIG. 24 is a graph showing that treatment with pLXIX-GAL4-IM and pAC3-GAL4BS-RLI increased tumor infiltration of lymphocytes at a 14 day time point, including CD3+ immune cell infiltration (3.0% vs. 20.0%, p=0.001) and CD8 T cell infiltration (0.8% vs. 8.0%, p=0.01), as described in the Examples.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

### INTRODUCTION

Replication-competent viral vectors, for example, retroviral vector (RRVs) have the ability to replicate and spread throughout dividing cells, including tumor cells. In addition, RRV stably integrate in cells that they infect, leading to persistent expression of the RRV genetic information by the infected cells. Additional non-viral genes can be placed into the RRV genetic information, leading to stable expression of these genes in addition to the viral genes necessary for replication.

Replication-deficient viral vectors, for example, retroviral vector (RDVs) lack some or all of the viral genes necessary for replication and, as a result, can deliver a larger non-viral genetic payload to tumor cells. However, they cannot replicate and lack the ability to spread through a tumor, which makes them ineffective in achieving therapeutic benefit in clinical trials, due to inadequate levels of gene delivery to tumors.

Infecting a cell with both a replication competent viral vector and a replication-deficient viral vector, for example, an RRV and an RDV simultaneously, or sequentially (but only if the cell is infected first with RDV), and then with RRV leads to production and spread of both viruses, because the viral proteins produced by RRV also allow the RDV to spread. However, RDV spread is limited, and generally, the RRV will rapidly outcompete the RDV and spread throughout a tumor more quickly.

Furthermore, any cells that are infected by the RRV first, lose the ability to subsequently become infected by another RDV derived from the same virus, a phenomenon known as superinfection resistance. This occurs because, when RRV infects a cell first, the virus naturally produces its envelope (env) protein as it replicates and assembles more virus particles in the infected cell. These envelope proteins endogenously engage and sequester the cellular proteins that serve as cell surface receptors, which are also needed for RDV to infect the cell. This mechanism blocks RDV from entering cells that have already been infected with RRV.

Alternatively, replicative spread can be achieved by using two RDV which are trans-complementary, or semi-replicative, i.e., the virus genome is split between the two vectors, and each RDV provides the components that the other lacks. Again, however, if the RDV that expresses the env protein infects a cell first, this will block the other RDV from entering the same cell, and terminate further replication.

The novel, coordinately-regulated viral gene delivery systems provided herein address these issues. For example, using the compositions and methods provided herein, an RRV can be engineered to depend on an RDV in order for the RRV to spread throughout a tumor. This is achieved by incorporating a nucleic acid encoding an activator in the RDV, which acts as an activator for the RRV, when both vectors have infected the same cell. Hence, the RRV can replicate only if the RDV is also present, and in turn, also helps the RDV replicate. This combination prevents the RRV from outcompeting the RDV since it is dependent on the RDV in order to replicate. In another embodiment, two RDVs that can trans-complement each other's replicative gene functions are coordinately regulated, such that an RDV expressing the env gene (which would normally monopolize cell receptors for the virus in the infected cell) is dependent on a second RDV to provide an activator, for example, a transcription factor that binds to a promoter in the first RDV and only then allows env gene transcription to proceed. Thus, superinfection resistance is prevented, and both RDVs, together, can express the full complement of virus genes, enabling both to replicate and spread through a tumor.

### Definitions

As used in this specification and the appended claims, the singular forms “a,” “an,” and “the” include plural reference unless the context clearly dictates otherwise.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of and “consisting of those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “retroviruses” are RNA viruses wherein the viral genome is RNA. When a host cell is infected with a retrovirus, the genomic RNA is reverse transcribed into a DNA intermediate which is integrated very efficiently into the chromosomal DNA of infected cells. The integrated DNA intermediate is referred to as a provirus. Retroviruses are enveloped single-stranded RNA viruses that typically infect mammals, such as, for example, bovines, monkeys, sheep, and humans, as well as avian species. Retroviruses are unique among RNA viruses in that their multiplication involves the synthesis of a DNA copy of the RNA which is then integrated into the genome of the infected cell.

The Retroviridae family consists of three groups: the spumaviruses (or foamy viruses) such as the human foamy virus (HFV); the lentiviruses, as well as visna virus of sheep; and the oncoviruses (although not all viruses within this group are oncogenic). The term “retrovirus” is used in its conventional sense to describe a genus of viruses containing reverse transcriptase. Retroviruses include lentiviruses. The lentiviruses include the “immunodeficiency viruses” which include human immunodeficiency virus (HIV) type 1 and type 2 (HIV-1 and HIV-2) and simian immunodeficiency virus (SIV). The oncoviruses are further subdivided into groups A, B, C and D on the basis of particle morphology, as seen under the electron microscope during viral maturation.

Retroviruses are defined by the way in which they replicate their genetic material. During replication the RNA is converted into DNA. Following infection of the cell a double-stranded molecule of DNA is generated from the two molecules of RNA which are carried in the viral particle by the molecular process known as reverse transcription. The DNA form becomes covalently integrated in the host cell genome as a provirus, from which viral RNAs are expressed with the aid of cellular and/or viral factors. The expressed viral RNAs are packaged into particles and released as infectious virion.

The retrovirus particle is composed of two identical RNA molecules. Each wild-type genome has a positive sense, single-stranded RNA molecule, which is capped at the 5′ end and polyadenylated at the 3′ tail. The diploid virus particle contains the two RNA strands complexed with gag proteins, viral enzymes (pol gene products) and host tRNA molecules within a ‘core’ structure of gag proteins. Surrounding and protecting this capsid is a lipid bilayer, derived from host cell membranes and containing viral envelope (env) proteins. The env proteins bind to a cellular receptor for the virus and the particle typically enters the host cell via receptor-mediated endocytosis and/or membrane fusion. After the outer envelope is shed, the viral RNA is copied into DNA by reverse transcription. This is catalyzed by the reverse transcriptase enzyme encoded by the pol region and uses the host cell tRNA packaged into the virion as a primer for DNA synthesis. In this way the RNA genome is converted into the more complex DNA genome. The double-stranded linear DNA produced by reverse transcription may, or may not, have to be circularized in the nucleus. The provirus now has two identical repeats at either end, known as the long terminal repeats (LTR). The termini of the two LTR sequences produces the site recognized by a pol product, the integrase protein, which catalyzes integration, such that the provirus is always joined to host DNA two base pairs (bp) from the ends of the LTRs. A duplication of cellular sequences is seen at the ends of both LTRs. Integration is thought to occur essentially at random within the target cell genome. However, by modifying the long-terminal repeats it is possible to control the integration of a retroviral genome.

Transcription, RNA splicing and translation of the integrated viral DNA is mediated by host cell proteins. Efficient infectious transmission of retroviruses requires the expression on the target cell of receptors which specifically recognize the viral envelope proteins, although viruses may use receptor-independent, nonspecific routes of entry at low efficiency. In addition, the target cell type must be able to support all stages of the replication cycle after virus has bound and penetrated.

As used herein, “a viral vector” refers to a gene therapy vector used to deliver a polynucleotide construct to a cell. It is understood that the term viral vector encompasses recombinant vector particles or virions (i.e., viral particles comprising at least one capsid or envelope protein and an encapsidated recombinant viral vector) and recombinant vector plasmids.

As used herein, a “recombinant viral vector” refers to a viral vector, for example, a retroviral vector comprising a nucleic acid sequence that is not normally present in the viral vector (i.e., a polynucleotide heterologous to the viral vector). Other vectors include, but are not limited to adenoviral vectors, adeno-associated viral vectors and herpes simplex vectors. In general, the heterologous nucleic acid is flanked by at least one, and generally by two, long terminal repeat sequences (LTRs), for example, a 5′ LTR and a 3′LTR. As used herein, the retroviral vector can be a derivative of a murine, simian or human retrovirus. Examples of retroviral vectors in which a transgene (e.g., a heterologous polynucleotide sequence) can be inserted include, but are not limited to lentivirus, Moloney murine leukemia virus (MoMuLV), Harvey murine sarcoma virus (HaMuSV), murine mammary tumor virus (MuMTV), Rous Sarcoma Virus (RSV), and foamy virus.

The term “nucleic acid” or “nucleotide” refers to deoxyribonucleic acids (DNA) or ribonucleic acids (RNA) and polymers thereof, for example, polynucleotides, in either single- or double-stranded form. The nucleic acid molecule may be derived from a variety of sources, including DNA, cDNA, synthetic DNA, RNA, or combinations thereof. Such nucleic acid sequences may comprise genomic DNA which may or may not include naturally occurring introns. Moreover, such genomic DNA may be obtained in association with promoter regions, introns, or poly A sequences. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or dcoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” or “transgene” can refer to the segment of DNA (e.g. a polynucleotide sequence) involved in producing or encoding a polypeptide chain. It may include regions preceding and following the coding region (leader and trailer) as well as intervening sequences (introns) between individual coding segments (exons). Alternatively, the term “gene” or “transgene” can refer to the segment of DNA involved in producing or encoding a non-translated RNA, such as an rRNA, tRNA, guide RNA (e.g., a single guide RNA), or micro RNA.

As used herein the phrase “heterologous” refers to what is not normally found in nature. The term “heterologous nucleotide sequence” refers to a nucleotide sequence not normally found in a given wild-type viral genome, or a cell in nature. As such, a heterologous nucleotide sequence may be: (a) foreign to its host cell (i.e., is exogenous to the cell) or viral genome; (b) naturally found in the host cell (i.e., endogenous) but present at an unnatural quantity in the cell (i.e., greater or lesser quantity than naturally found in the host cell); or (c) be naturally found in the host cell or viral genome but positioned outside of its natural locus.

As used herein, the term “activator” is a molecule, for example, a polypeptide or a polynucleotide sequence that activates expression of any of the polynucleotide sequences from a viral vector, e.g., a retroviral vector, described herein. In the compositions and methods provided herein, the activator activates expression of a polynucleotide on a different vector from the one that encodes the activator, i.e., is a trans-acting activator. In some embodiments, the activator activates expression of the polynucleotide by activating transcription or translation of the polynucleotide sequence. In instances where the activator activates transcription of the polynucleotide sequence, transcriptional activation can occur via binding of the activator to a regulator element, for example, a cis-acting regulatory element that controls expression of the polynucleotide. Cis-acting regulatory elements include, but are not limited to, a promoter sequence, an enhancer sequence and a repressor binding sequence.

In some examples, the activator is a transcriptional activator that binds to a promoter that controls expression of the polynucleotide sequence, thus activating transcription of the polynucleotide sequence. In some embodiments, the activator is a de-repressor that activates expression of a polynucleotide sequence by de-repressing or removing repression of transcription or translation of the polynucleotide sequence in a viral vector.

A “promoter” is defined as one or more a nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

A nucleic acid is “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation.

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

As used herein, the phrase “introducing” in the context of introducing a nucleic acid or a viral vector refers to the translocation of the nucleic acid sequence or viral vector from outside a cell to inside the cell. In some cases, introducing refers to infecting a cell or a population of cells with a viral vector or viral particle carrying one or more non-viral nucleic acids. In some cases, translocation of the nucleic acid from outside the cell to inside the nucleus of the cell occurs. Various methods of such translocation are contemplated, including but not limited to, viral infection, electroporation, transfection, transduction, contact with nanowires or nanotubes, receptor mediated internalization, translocation via cell penetrating peptides, liposome mediated translocation, and the like.

As used herein, the term “selectable marker” refers to a gene which allows selection of a host cell comprising a marker. The selectable markers may include, but are not limited to: fluorescent markers, luminescent markers and drug selectable markers, cell surface receptors, and the like. In some embodiments, the selection can be positive selection; that is, the cells expressing the marker are isolated from a population, e.g. to create an enriched population of cells expressing the selectable marker. Separation can be by any convenient separation technique appropriate for the selectable marker used. For example, if a fluorescent marker is used, cells can be separated by fluorescence activated cell sorting, whereas if a cell surface marker has been inserted, cells can be separated from the heterogeneous population by affinity separation techniques, e.g. magnetic separation, affinity chromatography, “panning” with an affinity reagent attached to a solid matrix, fluorescence activated cell sorting or other convenient technique.

As used herein, a “cell” can be in vivo, ex vivo or in vitro, and includes any cell or populations of cells that can be infected by a virus, (e.g., a retrovirus), for example, a human cell. As used herein, the term “cell” includes non-dividing cells, dividing cells, and cells exhibiting uncontrolled proliferation. A used herein, a non-dividing cell refers to a cell that does not go through mitosis. Dividing cells are cells that undergo active mitosis, or meiosis. Such dividing cells include stem cells, skin cells (e.g., fibroblasts and keratinocytes), gametes, and other dividing cells known in the art. Dividing cells include cells associated with cell proliferative disorders, for example, neoplastic cells.

Other cells that can be infected also include, but are not limited to, peripheral blood dendritic cells, follicular dendritic cells, B cells, natural killer cells, primary cells, hematopoietic cells, stem cells, eosinophils, precursor CD4⁺ bone marrow cells, immature thymic precursor cells. T cells, Langerhans cells, megakaryocytes, neurons, astrocytes, oligodendroglia, renal epithelial cells, cervical cells, rectal, bowel mucosal cells. Other cells and tissues from organs such as brain, liver, lungs, breast, ovaries, esophagus, skin, salivary glands, eyes, prostate, testes, and adrenals can also be infected.

As used herein the term “stem cells” includes multipotent, pluripotent, and totipotent stem cells. In some embodiments, the stem cell is a hematopoietic stem cell. As used herein, the phrase “hematopoietic stem cell” refers to a type of stem cell that can give rise to a blood cell. Hematopoietic stem cells can give rise to cells of the myeloid or lymphoid lineages, or a combination thereof.

As used herein, the phrase “hematopoietic cell” refers to a cell derived from a hematopoietic stem cell. The hematopoietic cell may be obtained or provided by isolation from an organism, system, organ, or tissue (e.g., blood, or a fraction thereof). Alternatively, an hematopoietic stem cell can be isolated and the hematopoietic cell obtained or provided by differentiating the stem cell. Hematopoietic cells include cells with limited potential to differentiate into further cell types. Such hematopoietic cells include, but are not limited to, multipotent progenitor cells, lineage-restricted progenitor cells, common myeloid progenitor cells, granulocyte-macrophage progenitor cells, or megakaryocyte-erythroid progenitor cells. Hematopoietic cells include cells of the lymphoid and myeloid lineages, such as lymphocytes, erythrocytes, granulocytes, monocytes, and thrombocytes. In some embodiments, the hematopoietic cell is an immune cell, such as a T cell, B cell, macrophage, a natural killer (NK) cell or dendritic cell. In some embodiments the cell is an innate immune cell.

As used herein, the phrase “primary” in the context of a primary cell is a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized directly without culturing or sub-culturing. In some cases, the primary cells are stimulated, activated, or differentiated. For example, primary T cells can be activated by contact with (e.g., culturing in the presence of) CD3, CD28 agonists, IL-2, IFN-γ, or a combination thereof.

The term “identity” or “substantial identity”, as used in the context of a polynucleotide sequence described herein (for example, SEQ ID NO: 1-13), refers to a sequence that has at least 60% sequence identity to a reference sequence. Alternatively, percent identity can be any integer from 60% to 100%. Exemplary embodiments include at least: 60%, 65%, 70%, 75%, 80%, 85%, 90%, 91%, 92%, 93%, 94%, 95%, 96%, 97%, 98%, or 99%, as compared to a reference sequence using the programs described herein; preferably BLAST using standard parameters, as described below.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, about 20 to 50, about 20 to 100, about 50 to about 200 or about 100 to about 150, in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands.

The BLAST algorithm also performs a statistical analysis of the similarity between two sequences (see, e.g., Karlin & Altschul, Proc. Nat′l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10-5, and most preferably less than about 10-20.

### Compositions

Provided herein are recombinant virus systems comprising two or more viral vectors, which are coordinately regulated. For example, two or more viral vectors, three or more viral vectors, four or more viral vectors, five or more viral vectors, etc., can be used in the systems provided herein. In some embodiment, the two or more, three or more, four or more, five or more viral vectors, etc., are adenoviral vectors or retroviral vectors, which are coordinately regulated.

In some embodiments, the infectious spread of a fully replication-competent retroviral vector (RRV) carrying a heterologous polynucleotide is regulated by a replication-defective retroviral (RDV) vector expressing an activator, for example, a transcriptional activator that activates expression of the heterologous polynucleotide by activating transcription or translation of the heterologous polynucleotide.

In other exemplary embodiments, a first RDV expressing a viral gene necessary for replication and a heterologous polynucleotide encoding a payload polypeptide, is regulated by a second RDV expressing an (i) activator; (ii) optionally, a second heterologous polynucleotide encoding a payload polypeptide; and (iii) complementary viral genes (i.e., complementary to the viral gene necessary for replication carried by the RDV) required for replication of both vectors.

Provided herein is a recombinant retrovirus system comprising: (a) a first retrovirus (i) encoding a first regulatory element operably linked to a nucleic acid encoding a first activator; and (ii) lacking a coding sequence for at least one viral protein required for replication such that the first retrovirus is a replication-deficient retrovirus (RDV); and (b) a second retrovirus comprising a nucleic acid comprising a first polynucleotide encoding the viral protein or proteins necessary for viral replication that are lacking in the first retrovirus, wherein the first polynucleotide is only expressed when the first activator activates expression of the first polynucleotide and/or its encoded viral protein(s).

In some embodiments, the second retrovirus comprises a second regulatory element operably linked to the first polynucleotide encoding a viral protein necessary for viral replication, and wherein the first activator activates transcription of the first polynucleotide by binding to the second regulatory element, for example, a promoter.

Also provided herein are systems, wherein the second retrovirus is a replication-deficient retrovirus (RDV) and encodes a third regulatory element operably linked to a nucleic acid encoding a second activator, and the first retrovirus comprises a nucleic acid comprising a second polynucleotide encoding a viral protein necessary for viral replication, wherein the second polynucleotide is only expressed when the second activator activates expression of the second polynucleotide, and wherein the first and second retroviruses can only replicate when the first and second activators are expressed.

In some embodiments, the first retrovirus comprises a fourth regulatory element operably linked to the second polynucleotide encoding a viral protein necessary for viral replication, and wherein the first activator activates transcription of the second polynucleotide by binding to the fourth regulatory element.

In any of the systems described herein, a regulatory element can be selected from the group consisting of a promoter, an enhancer, a promoter/enhancer combination or an epigenetic regulator. In some embodiments, epigenetic regulators can be used to modulate protein expression based on epigenetic markers. See, for example, Park et al. “Engineering epigenetic regulation using synthetic read-write modules,” Cell 176(1-2): 227-238 (2019) describing the use of M6a as an epigenetic marker. M6A is an epigenetic marker that is typically not found in humans. However, as described in Park et al., a synthetic “writer” can put an m6A mark on a specific DNA sequence and a synthetic “reader” can then specifically bind to the m6A sequence to induce gene expression.

In any of the systems described herein, the activator can activate expression of a polynucleotide sequence via transcriptional regulation or translational regulation (for example, an miRNA). In some embodiments, the activator activates expression by activating, i.e., increasing, transcription or translation of a polynucleotide sequence (for example, the first polynucleotide sequence). It is understood that, in the absence of the activator, the polynucleotide sequence is not expressed at all, or not expressed to any appreciable extent, such that the activator turns on expression of the polynucleotide.

In some embodiments, the activator is a transcriptional activator that binds to a promoter that is operably linked to the polynucleotide sequence to activate expression of the polynucleotide sequence. In some embodiments, the activator that binds to the promoter is a transcription factor. Exemplary activators include, but are not limited to, HIV-1 trans-activator protein (Tat), HIV-1 Rev, Gal4 or a fragment thereof, Gal4-VP16, and VP16-E2 and tetracycline transactivator protein. In some embodiments, a Tat-dependent replication competent virus stimulates or activates expression of the polynucleotide sequence via an RNA target sequence (TAR) included on an RDV (See, for example, FIG. 13). Exemplary activator sequences are set forth in Table 1. Sequences having at least 60%, 70%, 80%, 90%, 950, or 99% identity with any of the sequences provided in Table 1 are also provided. It is understood that any activator described herein that is expressed from a first vector, can bind to a corresponding site in a second vector, of any viral vector system described herein, to activate expression of one or more polynucleotides in the second vector.

TABLE 1
Exemplary Activator Sequences

| | | |
|---|---|---|
| SEQ ID NO: 7 | GAL4/VP16 | ATGAAGCTTCTCTCTAGCATTG |
| | | AACAGGCATGTGATATCTGCC |
| | | GACTCAAGAAACTAAAATGCT |
| | | CCAAGGAGAAGCCAAAGTGTG |
| | | CCAAGTGCTTGAAAAATAACT |
| | | GGGAGTGTCGCTACAGTCCCA |
| | | AGACCAAGCGTTCTCCGCTGA |
| | | CTCGGGCTCATCTCACCGAAG |
| | | TCGAGAGCCGCCTGGAACGTT |
| | | TAGAGCAATTGTTCCTCCTCAT |
| | | CTTCCCCCGGGAAGATCTTGA |
| | | CATGATCCTTAAGATGGACTC |
| | | CTTGCAGGATATCAAGGCCCT |
| | | CTTGACCGGGCTATTCGTTCA |
| | | AGACAATGTCAACAAGGACGC |
| | | AGTCACAGACCGCCTTGCGAG |
| | | CGTGGAGACTGATATGCCACT |
| | | GACGCTAAGGCAGCACAGAAT |
| | | TTCAGCCACAAGTAGTTCAGA |
| | | AGAATCTTCCAATAAAGGGCA |
| | | GCGGCAATTGACTGTCTCCCC |
| | | AGAGTTCCCTGCTGACGCCTT |
| | | GGATGATTTCGACCTCGACAT |
| | | GCTTCCAGCAGATGCTCTTGA |
| | | TGACTTTGATTTGGATATGCT |
| SEQ ID NO: 8 | HIV-1 Trans-Activator | ATGGAGCCAGTAGATCCTAGA |
| | Protein (Tat) NCBI | CTAGAGCCCTGGAAGCATCCA |
| | Reference Sequence: | GGAAGTCAGCCTAAAACTGCT |
| | NC_001802.1 | TGTACCAATTGCTATTGTAAA |
| | | AAGTGTTGCTTTCATTGCCAA |
| | | GTTTGTTTCATAACAAAAGCC |
| | | TTAGGCATCTCCTATGGCAGG |
| | | AAGAAGCGGAGACAGCGACG |
| | | AAGAGCTCATCAGAACAGTCA |
| | | GACTCATCAAGCTTCTCTATCA |
| | | AAGCAACCCACCTOCCAACCC |
| | | CGAGGGGACCCGACAGGCCCG |
| | | AAGGAATAG |
| SEQ ID NO: 9 | HIV-1 REV NCBI Reference | ATGGCAGGAAGAAGCGGAGA |
| | Sequence: NC_001802.1 | CAGCGACGAAGAGCTCATCAG |
| | | AACAGTCAGACTCATCAAGCT |
| | | TCTCTATCAAAGCAACCCACC |
| | | TCCCAACCCCGAGGGGACCCG |
| | | ACAGGCCCGAAGGAATAGAA |
| | | GAAGAAGGTGGAGAGAGAGA |
| | | CAGAGACAGATCCATTCGATT |
| | | AGTGAACGGATCCTTGGCACT |
| | | TATCTGGGACGATCTGCGGAG |
| | | CCTGTGCCTCTTCAGCTACCAC |
| | | CGCTTGAGAGACTTACTCTTG |
| | | ATTGTAACGAGGATTGTGGAA |
| | | CTTCTGGGACGCAGGGGGTGG |
| | | GAAGCCCTCAAATATTGGTGG |
| | | AATCTCCTACAGTATTGGAGT |
| | | CAGGAACTAAAGAATAG |
| SEQ ID NO: 10 | TRANSACTIVATOR rTTA | ATGTCTAGACTGGACAAGAGC |
| | (Reverse Tetracycline | AAAGTCATAAACGGAGCTCTG |
| | Controlled Transactivator) | GAATTACTCAATGGTGTCGGT |
| | | ATCGAAGGCCTGACGACAAGG |
| | | AAACTCGCTCAAAAGCTGGGA |
| | | GTTGAGCAGCCTACCCTGTAC |
| | | TGGCACGTGAAGAACAAGCGG |
| | | GCCCTGCTCGATGCCCTGCCA |
| | | ATCGAGATGCTGGACAGGCAT |
| | | CATACCCACTTCTGCCCCCTGG |
| | | AAGGCGAGTCATGGCAAGACT |
| | | TTCTGCGGAACAACGCCAAGT |
| | | CATACCGCTGTGCTCTCCTCTC |
| | | ACATCGCGACGGGGCTAAAGT |
| | | GCATCTCGGCACCCGCCCAAC |
| | | AGAGAAACAGTACGAAACCCT |
| | | GGAAAATCAGCTCGCGTTCCT |
| | | GTGTCAGCAAGGCTTCTCCCT |
| | | GGAGAACGCACTGTACGCTCT |
| | | GTCCGCCGTGGGCCACTTTAC |
| | | ACTGGGCTGCGTATTGGAGGA |
| | | ACAGGAGCATCAAGTAGCAAA |
| | | AGAGGAAAGAGAGACACCTA |
| | | CCACCGATTCTATGCCCCCACT |
| | | TCTGAGACAAGCAATTGAGCT |
| | | GTTCGACCGGCAGGGAGCCGA |
| | | ACCTGCCTTCCTTTTCGGCCTG |
| | | GAACTAATCATATGTGGCCTG |
| | | GAGAAACAGCTAAAGTGCGA |
| | | AAGCGGCGGGCCGACCGACGC |
| | | CCTTGACGATTTTGACTTAGAC |
| | | ATGCTCCCAGCCGATGCCCTT |
| | | GACGACTTTGACCTTGATATG |
| | | CTGCCTGCTGACGCTCTTGAC |
| | | GATTTTGACCTTGACATGCTCC |
| | | CCGGGTAA |
| SEQ ID NO: 11 | HIV-1 Trans-Activation | GGTCTCTCTGGTTAGACCAGA |
| | Response Element (TAR) | TCTGAGCCTGGGAGCTCTCTG |
| | Hairpin | GCTAACTAGGGAACC |

In some embodiments, the activator is a de-repressor that activates expression of a polynucleotide sequence by de-repressing or inhibiting a repressive biological event. For example, and not to be limiting, the de-repressor can be an miRNA sponge encoded by the first retroviral vector that interacts with (e.g., binds to) an miRNA expressed by the second retroviral vector, that represses expression and/or replication of the second retroviral vector. In the absence of the miRNA sponge, expression of the miRNA encoded by the second retroviral vector represses replication of the second retroviral vector. However, when an miRNA sponge is expressed from the first retroviral vector, the sponge acts to bind the miRNA that is normally repressed expression from the second retroviral vector, such that expression and replication of the second retroviral vector, in addition to replication from the first retroviral vector, can proceed. See, for example, Ebert and Sharp “MicroRNA sponges: Progress and possibilities” RNA 16(11): 2043-2050 (2010); and Tay et al. “Using artificial microRNA sponges to achieve microRNA loss-of-function in cancer cells,” Advanced Drug Delivery Reviews 81: 117-127 (2015).

In the compositions provided herein, when the viral vector, for example, a retroviral vector lacks at least one viral protein required for replication, the virus, for example, the retrovirus, can only replicate when combined with a viral vector, for example, a retroviral vector that comprises one or more viral proteins necessary for replication. In some embodiments, the first viral vector comprises one or more viral proteins necessary for viral replication, and the second viral vector comprises one or more viral proteins necessary for viral replication that are not included in the first viral vector. As used herein, “a viral protein necessary for replication” can be a Gag, Env, Pol. Rev or Tat viral protein. In some embodiments, this term refers to the Gag, Env or Pol retroviral proteins. For example, in some embodiments, the first retroviral vector (RDV) comprises a nucleic acid sequence encoding a Gag protein; and the second retroviral vector comprises (i) a nucleic acid sequence encoding an envelope (Env) protein, and (ii) a nucleic acid sequence encoding a retroviral Pol protein, such that when the vectors are combined, all of the viral proteins necessary for replication allow replication of the first and second retroviral vector. In another example, the first retrovirus is an RDV that does not comprise a viral protein necessary for viral replication and the second retrovirus is a RRV encoding all viral proteins necessary for viral replication (i.e., Gal. Pol and Env proteins). Also contemplated herein are modified viral proteins necessary for viral replication that can be used to target the retrovirus to certain cell or tissue types. For example, the Env protein sequence can be modified to include a target-specific ligand (i.e., an antibody, a receptor or receptor ligand) that binds to a particular cell or tissue type. In addition, tissue specific synthetic signaling proteins, such as SynNotch (Morsut et al. “Engineered Customized Cell Sensing and Response Behaviors using Synthetic Note Receptors,” Cell 164: 780-791 (2016)) could be used to control replication of the system and ensure tissue/target specific replication.

In any of the compositions provided herein, the promoter (for example, the first, second, third, or fourth promoters in any of the vectors provided herein) can be a constitutive promoter (e.g., SV40, EF1A, RSV, CMV, etc.) or an inducible promoter (e.g., tetracycline (Iida et al. J. Virol., 70(9): 6054-9), GAL4 target upstream activating sequence (Osterwalder et al., PNAS 98(22): 12596-12601 (2001), Cumate inducible expression system (Seo and Dannert, Appl. Microbiol. Biotechnol. 103(1): 303-313 (2019)). In any of the retroviral vectors provided herein, a promoter can be contained within an LTR sequence, for example, in the 5′ or 3′ LTR sequence or adjacent to the heterologous polynucleotide sequence, for example, adjacent to the 3′ end of a polynucleotide sequence (for example, the first polynucleotide sequence, the second polynucleotide sequence, the third polynucleotide sequence etc.). Any of the 3′LTR sequences described herein, for example, a retroviral 3′LTR sequence (e.g., a MMLV 3′LTR sequence) can be modified to reduce or disrupt native promoter function in the 3′LTR, for example, by deleting one or more sequences in the 3′LTR and/or inserting one or more sequences, for example, one or more nucleic acid sequences comprising a GAL4 binding site, in the 3′LTR. An exemplary GAL4 binding site is provided herein as consensus sequence (CGGNIICCG), wherein N is any nucleotide (A,C, T or G), or SEQ ID NO: 4 (cggagtactgtcctccgagcgg), as shown in SEQ ID NO: 2 and SEQ ID NO: 3. Exemplary sequences comprising one or more GAL4 binding sites are provided herein as SEQ ID NO: 2 and SEQ ID NO: 3. In some embodiments, one or more LTRs of the vectors, for example, the 3LTR and/or the 5′LTR, can comprise one, two, three, four, five, six, seven, eight, nine, ten, or more binding sites, for example, a GAL4 binding site, for an activator depending on the desired level of expression of one or more polynucleotides in a particular cell, tissue or organ. As described in the Examples, in some embodiments, the 3′ LTR and the 5′LTR comprise the same sequence once an infectious particle comprising the viral vector is produced.

The promoter can also be a cell-specific or tissue-specific promoter. When using a cell- or tissue-specific promoter, viral replication occurs primarily, but not exclusively, in a particular cell or tissue. For example, viral replication can occur in at least 90%, 95%, or 99% of the targeted cell or tissue. It will be understood, however, that tissue-specific promoters may have a detectable amount of background or base activity in those tissues where they are mostly silent. The degree to which a promoter is selectively activated in a target tissue can be expressed as a selectivity ratio (activity in a target tissue/activity in a control tissue). In this regard, a tissue-specific promoter useful in the practice of the present invention typically has a selectivity ratio of greater than about 5. Preferably, the selectivity ratio is greater than about 15.

Examples of tissue-specific promoters include, but are not limited to, liver-specific promoters (e.g., APOA2, SERPINA1, CYP3A4, MIR122), pancreatic-specific promoters (e.g., insulin, insulin receptor substrate 2, pancreatic and duodenal homeobox 1, Aristaless-like homeobox 3, and pancreatic polypeptide), cardiac-specific promoters (e.g., myosin, heavy chain 6, myosin, light chain 2, troponin I type 3, natriuretic peptide precursor A, solute carrier family 8), central nervous system promoters (e.g., glial fibrillary acidic protein, intemexin neuronal intermediate filament protein, Nestin, myelin-associated oligodendrocyte basic protein, myelin basic protein, tyrosin hydroxylase, and Forkhead box A2), skin-specific promoters (e.g., Filaggrin, Keratin 14 and transglutaminase 3), pluripotent and embryonic germ layer promoters (e.g., POU class 5 homeobox 1, Nanog homeobox, Nestin, and MicroRNA 122). In some embodiments, the tissue-specific promoter is in the U3 region of the LTR of the retroviral genome, including for example cell- or tissue-specific promoters and enhancers to neoplastic cells (e.g., tumor cell-specific enhancers and promoters), and inducible promoters (e.g., tetracycline).

In some embodiments, the first and/or second retrovirus further comprises a heterologous expression cassette comprising a payload promoter operably linked to a payload polynucleotide sequence. In some embodiments, since an RDV can include larger inserts, as compared to the RRV, the RDV comprises the heterologous expression cassette comprising a payload promoter operably linked to a payload polynucleotide sequence. In the systems provided herein, the defective virus can comprise an insert as large as, about 8 to about 10 kb, and the replication-competent virus can comprise an insert as large as about 1.3 kb to 1.5 kb. In some embodiments, the payload polynucleotide sequence encodes an antisense molecule, a ribozyme, a therapeutic protein, an immunomodulator, an antibody, an enzyme, a prodrug activator, or a cytotoxic protein. Other proteins of interest include, but are not limited to, Cas activator proteins, Cas phi, immunomodulatory proteins (for example, CD40L, 4-1BBL, OX40L, GITRL, IL-15, IL-12, scFvs (anti PD1, anti lag3, anti Tim3, etc.), FLT3L, GMCSF, or VEGF-C), synthetic immunomodulators (BiTEs, surface T cell engagers, etc.), suicide genes (for example, gamma-CD, NAO and thymidine kinase) and synthetic signaling molecules, for example, SynNotch, can also be expressed.

In some embodiments, the prodrug activator is encoded by a suicide gene, for example, thymidine kinase, cytidine deaminase or purine nucleoside phosphorylase (PNP). An example of a system that can be used to deliver an immunomodulator is shown in FIG. 15. In this example, IL-15 is encoded by the replication competent viral vector and the immunomodulator(s) is encoded by the replication deficient viral vector. The viral vector systems described herein can also be used to edit the genome of any host or target cell.

An example, of a system that can be used to deliver a guide RNA and guided gene editing nuclease (e.g., Cas9), as part of a CRISPR/Cas system, is shown in FIG. 15. In this example, the guide RNA is encoded by the replication competent viral vector (e.g., replication competent retroviral vector) of the system, and Cas9 is encoded by the replication deficient viral vector (e.g., replication deficient retroviral vector) of the system. In some embodiments multiple gRNAs are used to target more than one site in the genome of a cell(s). Multiple guide RNAs can be expressed by, for example, encoding each gRNA under the control of an individual promoter (e.g., U6, H1). These gRNA expression cassettes can be incorporated into the RDV and/or the RRV.

Alternative methods can be employed to multiplex gRNA sequences within an individual vector, these include, but are not limited to the generation of crRNA or gRNA arrays flanked by different sequences (e.g., direct repeats required for processing of pre-crRNA, self-cleaving sequences such as HDV ribozymes, Csy4 recognition sites, tRNAs). These gRNA/crRNA arrays will then be processed by endogenous (RNaseIII, RNaseP, RNase Z) or exogenous (Csy4) proteins enabling multiple loci to be edited simultaneously. See, for example, Feng and Yang, “Efficient expression of multiple guide RNAs for CRISPR/Cas Genome Editing,” aBIOTECH 1: 123-134 (2020)).

In some embodiments, a multiple gRNA configuration could be used for sequential, coordinated regulation of RDV/RRV expression: For example, a gRNA sequence, included with a sequence encoding Cas9 in the RDV, could be used to knock out an endogenous gene encoding a transcriptional repressor protein, whose cognate binding site sequence could be built into the RRV 3′ LTR promoter (and therefore copied over to the 5′ LTR). Therefore, only when the repressor protein is knocked out, by virtue of the RDV already being in the target cell first, could any RRV that was introduced into the same target cell (after, for example, being expressed from an adjacent cell) then be capable of expressing itself, thereby alleviating superinfection resistance, i.e., alleviating superinfection resistance by alleviating transcriptional repression.

The “CRISPR/Cas” system refers to a widespread class of bacterial systems for defense against foreign nucleic acid. CRISPR/Cas systems are found in a wide range of cubacterial and archaeal organisms. CRISPR/Cas systems include type I, II, and III sub-types. Wild-type type II CRISPR/Cas systems utilize an RNA-mediated nuclease, for example, Cas9, in complex with guide and activating RNA to recognize and cleave foreign nucleic acid. Guide RNAs having the activity of both a guide RNA and an activating RNA are also known in the art. In some cases, such dual activity guide RNAs are referred to as a single guide RNA (sgRNA).

Cas9 homologs are found in a wide variety of eubacteria, including, but not limited to bacteria of the following taxonomic groups: Actinobacteria, Aquficae, Bacteroidetes-Chlorobi, Chlamydiae-Verrucomicrobia, Chiroflexi, Cyvnobacteria, Firmicutes, Proteobacteria. Spirochaeles, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep. 24; 110(39):15644-9; Sampson et al.. Nature. 2013 May 9:497(7448):254-7; and Jinek, et al.. Science. 2012 Aug. 17; 337(6096):816-21. Variants of any of the Cas9 nucleases provided herein can be optimized for efficient activity or enhanced stability in the host cell. Thus, engineered Cas9 nucleases are also contemplated. See, for example, “Slaymaker et al., “Rationally engineered Cas9 nucleases with improved specificity.” Science 351 (6268): 84-88 (2016)).

As used herein, the term “Cas9” refers to an RNA-mediated nuclease (e.g., of bacterial or archeal orgin, or derived therefrom). Exemplary RNA-mediated nucleases include the foregoing Cas9 proteins and homologs thereof. Other RNA-mediated nucleases include Cpf1 (See, e.g., Zetsche et al., Cell, Volume 163, Issue 3, p759-771, 22 Oct. 2015) and homologs thereof. As used herein, the term “ribonucleoprotein” complex and the like refers to a complex between a targeted nuclease, for example. Cas9, and a crRNA (e.g., guide RNA or single guide RNA), the Cas9 protein and a trans-activating crRNA (tracrRNA), the Cas9 protein and a guide RNA, or a combination thereof (e.g., a complex containing the Cas9 protein, a tracrRNA, and a crRNA guide RNA). It is understood that in any of the embodiments described herein, a Cas9 nuclease can be substituted with a Cpf1 nuclease or any other guided nuclease.

In some embodiments, the CRISPR-associated endonuclease is a catalytically impaired nuclease. As used throughout, “catalytically impaired” refers to decreased CRISPR-associated endonuclease enzymatic activity for cleaving one or both strands of DNA. Examples of catalytically impaired CRISPR-associated endonuclease, include but are not limited to, catalytically impaired Cas9, catalytically impaired Cpf1, and catalytically impaired C2c2. In some instances, the catalytically impaired CRISPR-associated endonuclease is a catalytically impaired Cas9, for example Cas9 D10A, which cleaves or nicks only one strand of DNA. In some instances, the CRISPR-associated endonuclease may be a catalytically impaired CRISPR-associated endonuclease, wherein the endonuclease cannot cleave both strands of a double-stranded DNA molecule, i.e., cannot make a double-stranded break. Modifications include, but are not limited to, altering one or more amino acids to inactivate the nuclease activity or the nuclease domain. For example, and not to be limiting, D10A and/or H840A mutations can be made in Cas9 from Streptococcus pyogenes to reduce or inactivate Cas9 nuclease activity. Other modifications include removing all or a portion of the nuclease domain of Cas9, such that the sequences exhibiting nuclease activity are absent from Cas9. Accordingly, a catalytically impaired Cas9 may include polypeptide sequences modified to reduce nuclease activity or removal of a polypeptide sequence or sequences to reduce nuclease activity. The catalytically impaired Cas9 retains the ability to bind to DNA even though the nuclease activity has been inactivated. Accordingly, a catalytically impaired Cas9 includes the polypeptide sequence or sequences required for DNA binding but includes modified nuclease sequences or lacks nuclease sequences responsible for nuclease activity. It is understood that similar modifications can be made to reduce nuclease activity in other site-directed nucleases, for example in Cpf1 or C2c2. In some examples, the Cas9 protein is a full-length Cas9 sequence from S. pyogenes lacking the polypeptide sequence of the RuvC nuclease domain and/or the HNH nuclease domain and retaining the DNA binding function. In other examples, the Cas9 protein sequences have at least 30%, 40%, 50%, 60%, 70%, 80%, 90%, 95%, 98% or 99% identity to Cas9 polypeptide sequences lacking the RuvC nuclease domain and/or the HNH nuclease domain and retains DNA binding function. Any of the catalytically impaired RNA-guided nucleases described herein can be used to inhibit transcription of a gene. In some embodiments, the dCas9 is fused with a transcriptional repressor or transcriptional activator, for transcriptional repression or activation of a gene(s) targeted by one or more gRNAs.

In some embodiments, the viral vector, for example, a retroviral vector, contains an IRES comprising a cloning site for insertion of one or more payload polynucleotide sequences. Accordingly, a heterologous polynucleotide sequence encoding a desired polypeptide may be operably linked to the IRES. An example of polynucleotide sequence which may be operably linked to the IRES include green fluorescent protein (GFP) or a selectable marker gene. Marker genes are utilized to assay for the presence of the vector, and thus, to confirm infection and integration. Typical selection genes encode proteins that confer resistance to antibiotics and other toxic substances, e.g., histidinol, puromycin, hygromycin, neomycin, methotrexate, and other reporter genes known in the art. Other polynucleotide sequences which may be linked to the IRES include, for example, polynucleotide sequences that encode a polypeptide selected from the group consisting of a therapeutic protein, a prodrug activator, an enzyme, an antibody, and a cytotoxic protein. In some embodiments, the prodrug activator is thymidine kinase, cytidine deaminase or purine nucleoside phosphorylase (PNP).

In some embodiments, the components of the viral vector, e.g., a retroviral vector, are expressed in multicistronic fashion, by including one or more self-cleaving peptides in between two or more nucleic acids to be expressed as a multicistronic, for example, a bicistronic sequence. Examples of self-cleaving peptides include, but are not limited to, self-cleaving viral 2A peptides, for example, a porcine teschovirus-1 (P2A) peptide, a Thosea asigna virus (T2A) peptide, an equine rhinitis A virus (E2A) peptide, or a foot-and-mouth disease virus (F2A) peptide. Self-cleaving 2A peptides allow expression of multiple gene products from a single construct. (See, for example, Chng et al. “Cleavage efficient 2A peptides for high level monoclonal antibody expression in CHO cells.” MAbs 7(2): 403-412 (2015)). In some embodiments, the nucleic acid construct comprises two or more self-cleaving peptides. In some embodiments, the two or more self-cleaving peptides are all the same. In other embodiments, at least one of the two or more self-cleaving peptides is different.

The first virus and/or the second virus of any of the systems described herein, either as nucleic acid vectors, or viral particles, can be formulated as a pharmaceutical composition. In some embodiments, the pharmaceutical composition can further comprise a carrier. The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject. The compositions will include a therapeutically effective amount of one or more retroviruses described herein in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained. Such pharmaceutically acceptable carriers include sterile biocompatible pharmaceutical carriers, including, but not limited to, saline, buffered saline, artificial cerebral spinal fluid, dextrose, and water.

### Methods of Making Retroviral Systems

Provided herein is a method for making a recombinant virus system comprising: (a) transfecting a first suitable host cell with the first viral vector of any of the virus systems described herein; (b) transfecting a second suitable host cell with the second viral vector of any of the virus systems described herein; and (c) recovering the first and second viruses. In some embodiments, the first and second virus and transfected into the same suitable host cell. In some embodiments, the first and second viruses and retroviruses.

### Methods

Also provided are methods for using any of the coordinately-regulated viral vector systems provided herein to efficiently deliver transgenes to tumors in vivo for therapeutic, diagnostic, and theranostic purposes. In some embodiments, any of the systems describe herein can be used for diagnostic purposes by introducing transgenes that are only expressed under certain conditions (for example, presence of high tumor infiltrating T cells). These transgenes could provide a non-invasive detectable signal (for example, fluorescent protein expression). In a theranostic situation, the viral system could be constructed to sense specific changes in the tumor microenvironment (for example, increased myeloid cell infiltration) leading to expression of therapeutic genes (for example, myelotoxic genes).

Provided herein is a method for transducing a target cell with a replicating virus system comprising contacting the target cell with the first virus and second virus of any of the virus systems provided herein. In some embodiments, the virus system is a retrovirus system, wherein the first virus and the second virus are retroviruses. In any of the methods for transfecting a target cell, viral DNA that can produce viral particles in the transfected cells (for example, a recombinant retroviral polynucleotide or viral particles comprising a recombinant retroviral polynucleotide) can be delivered to the cells. In some embodiments, the cell is infected with the first virus particles and second virus particles of any of the virus systems provided herein. In some embodiments, the cell is a mammalian cell. In any of the methods provided herein, the cell can be contacted in vitro, ex vivo or in vivo.

Also provided are methods for treating a disease in a subject in need thereof comprising administering a therapeutically effective amount of any of the systems or pharmaceutical compositions provided herein to the subject. In some methods, cells are removed from the subject, modified ex vivo using any of the recombinant virus systems described herein and administered to the patient after modification. In some methods, the modified cells are expanded before administration to the patient.

In any of the methods provided herein, the subject can be a subject diagnosed with a disease, for example, a cell proliferative disorder. In any of the methods of treatment provided herein, the first and second virus of the system are administered simultaneously or sequentially to the subject. Any of the methods of treatment provided herein can be used to deliver a polynucleotide encoding any of the payload polypeptides described herein to the subject. The polypeptide can be selected from the group consisting of a therapeutic protein, a prodrug activator, an enzyme, an antibody, and a cytotoxic protein. In some embodiments, the prodrug activator is thymidine kinase, cytidine deaminase or purine nucleoside phosphorylase (PNP). In cases where a prodrug activator is used, a nontoxic prodrug can be administered to the subject, such that when the activator is expressed, conversion of the nontoxic prodrug into a toxic drug, i.e. cell-killing drug, takes place.

In some methods, the virus system is a recombinant retrovirus system comprising: (a) a first retrovirus (i) encoding a first regulatory element operably linked to a nucleic acid encoding a first activator; and (ii) lacking a coding sequence for at least one viral protein required for replication such that the first retrovirus is a replication-deficient retrovirus (RDV); and (b) a second retrovirus comprising a nucleic acid comprising a first polynucleotide encoding the viral protein or proteins necessary for viral replication that are lacking in the first retrovirus, wherein the first polynucleotide is only expressed when the first activator expression of the first polynucleotide and/or its encoded viral protein(s).

In some embodiments, the second retrovirus comprises a second regulatory element operably linked to the first polynucleotide encoding a viral protein necessary for viral replication, and wherein the first activator activates transcription of the first polynucleotide by binding to the second regulatory element, for example, a promoter.

In some embodiments, the second retrovirus is a replication-deficient retrovirus (RDV) and encodes a third regulatory element operably linked to a nucleic acid encoding a second activator; and the first retrovirus comprises a nucleic acid comprising a second polynucleotide encoding a viral protein necessary for viral replication, wherein the second polynucleotide is only expressed when the second activator activates expression of the second polynucleotide, and wherein the first and second retroviruses can only replicate when the first and second activators are expressed. In some embodiments, the first retrovirus comprises a fourth regulatory element operably linked to the second polynucleotide encoding a viral protein necessary for viral replication, and wherein the first activator activates transcription of the second polynucleotide by binding to the fourth regulatory element.

In some embodiments, the first and/or second retrovirus further comprises a heterologous expression cassette comprising a payload promoter operably linked to a payload polynucleotide sequence. In some embodiments, the payload polynucleotide sequence encodes an antisense molecule, a ribozyme, a therapeutic protein, an immunomodulator, an antibody, an enzyme, a prodrug activator, or a cytotoxic protein. Other proteins of interest include, but are not limited to, Cas proteins, Cas activator proteins, Cas phi, immunomodulatory proteins (for example, CD40L, 4-1BBL, OX40L, GITRL, IL-15, IL-12, scFvs (anti PD1, anti lag3, anti Tim3, etc.), FLT3L, GMCSF, or VEGF-C), synthetic immunomodulators (BiTEs, surface T cell engagers, etc.), suicide genes (for example, gamma-CD, NAO and thymidine kinase) and synthetic signaling molecules, for example, SynNotch, can also be expressed.

In some embodiments, the prodrug activator is encoded by a suicide gene, for example, thymidine kinase, cytidine deaminase or purine nucleoside phosphorylase (PNP).

“Treating” refers to any indicia of success in the treatment or amelioration or prevention of the disease, condition, or disorder, including any objective or subjective parameter such as abatement; remission; diminishing of symptoms or making the disease condition more tolerable to the patient; slowing in the rate of degeneration or decline; preventing a relapse, or making the final point of degeneration less debilitating. For example, a method for treating cancer is considered to be a treatment if there is a 10% reduction in one or more symptoms of the cancer in a subject as compared to a control. Thus the reduction can be a 10%, 20%, 30%, 40/o, 50%, 60%, 70%, 80%, 90%, 100%, or any percent reduction in between 10% and 100% as compared to native or control levels. It is understood that treatment does not necessarily refer to a cure or complete ablation of the disorder or symptoms of the disorder.

Any of the methods provided herein can be used to treat a cell proliferative disorder. The term “cell proliferative disorder” refers to a condition characterized by an abnormal number of cells. The condition can include both hypertrophic (the continual multiplication of cells resulting in an overgrowth of a cell population within a tissue) and hypotrophic (a lack or deficiency of cells within a tissue) cell growth or an excessive influx or migration of cells into an area of a body. The cell populations are not necessarily transformed, tumorigenic or malignant cells, but can include normal cells as well.

In some cases, the cell proliferative disorder is cancer. As used herein, cancer is a disease characterized by the rapid and uncontrolled growth of aberrant cells. Cancer cells can spread locally or through the bloodstream and lymphatic system to other parts of the body. The cancer can be a solid tumor. In some embodiments, the cancer is a blood or hematological cancer, such as a leukemia (e.g., acute leukemia; acute lymphocytic leukemia, acute myelocytic leukemias, such as myeloblastic, promyclocytic, myelomonocytic, monocytic, erythroleukemia leukemias and myelodysplastic syndrome; chronic myelocytic (granulocytic) leukemia; chronic lymphocytic leukemia; hairy cell leukemia), polycythemia vera, or lymphomas (e.g., Hodgkin's disease or non-Hodgkin's disease lymphomas (e.g., diffuse anaplastic lymphoma kinase (ALK) negative, large B-cell lymphoma (DLBCL); diffuse anaplastic lymphoma kinase (ALK) positive, large B-cell lymphoma (DLBCL); anaplastic lymphoma kinase (ALK) positive, ALK+anaplastic large-cell lymphoma (ALCL), acute myeloid lymphoma (AML))), multiple myelomas (e.g., smoldering multiple myeloma, non-secretory mycloma, osteosclerotic myeloma, plasma cell leukemia, solitary plasmacytoma and extramedullary plasmacytoma). Waldenstrom's macroglobulinemia, monoclonal gammopathy of undetermined significance, benign monoclonal gammopathy and heavy chain disease. Solid tumors include, by way of example, bone and connective tissue sarcomas (e.g., bone sarcoma, ostcosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma), brain tumors (e.g., glioma, glioblastoma, astrocytoma, brain stem glioma, ependymoma, oligodendroglioma, nonglial tumor, acoustic neurinoma, craniopharyngioma, medulloblastoma, meningioma, pineocytoma, pincoblastoma, primary brain lymphoma), breast cancer (e.g., adenocarcinoma, lobular (small cell) carcinoma, intraductal carcinoma, medullary breast cancer, mucinous breast cancer, tubular breast cancer, papillary breast cancer, Paget's disease, and inflammatory breast cancer), adrenal cancer (e.g., pheochromocytoma and adrenocortical carcinoma), thyroid cancer (e.g., papillary or follicular thyroid cancer, medullary thyroid cancer and anaplastic thyroid cancer), pancreatic cancer (e.g., insulinoma, gastrinoma, glucagonoma, vipoma, somatostatin-secreting tumor, and carcinoid or islet cell tumor), pituitary cancers (e.g., Cushing's disease, prolactin-secreting tumor, acromegaly, and diabetes insipidus), eye cancers (e.g., ocular melanoma such as iris melanoma, choroidal melanoma, and ciliary body melanoma, and retinoblastoma), vaginal cancers (e.g., squamous cell carcinoma, adenocarcinoma, and melanoma), vulvar cancer (e.g., squamous cell carcinoma, melanoma, adenocarcinoma, basal cell carcinoma, sarcoma, and Paget's disease), cervical cancers (e.g., squamous cell carcinoma and adenocarcinoma), uterine cancers (e.g., endometrial carcinoma and uterine sarcoma), ovarian cancers (e.g., ovarian epithelial carcinoma, borderline tumor, germ cell tumor, and stromal tumor), esophageal cancers (e.g., squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma), stomach cancers (e.g., adenocarcinoma, fungating (polypoid), ulcerating, superficial spreading, diffusely spreading, malignant lymphoma, liposarcoma, fibrosarcoma, and carcinosarcoma), colon cancers, rectal cancers, liver cancers (e.g., hepatocellular carcinoma and hepatoblastoma), gallbladder cancers (e.g., adenocarcinoma), cholangiocarcinomas (papillary, nodular, and diffuse), lung cancers (e.g., non-small cell lung cancer, squamous cell carcinoma (epidermoid carcinoma), adenocarcinoma, large-cell carcinoma and small-cell lung cancer), testicular cancers (e.g., germinal tumor, seminoma, anaplastic, classic (typical), spermatocytic, nonseminoma, embryonal carcinoma, teratoma carcinoma, choriocarcinoma (yolk-sac tumor)), prostate cancers (e.g., adenocarcinoma, leiomyosarcoma, and rhabdomyosarcoma), penile cancers, oral cancers (e.g., squamous cell carcinoma), basal cancers, salivary gland cancers (e.g., adenocarcinoma, mucoepidermoid carcinoma, and adenoidcystic carcinoma), esopharyngeal cancers (e.g., squamous cell cancer and verrucous cancer), skin cancers (e.g., basal cell carcinoma, squamous cell carcinoma and melanoma, superficial spreading melanoma, nodular melanoma, lentigo malignant melanoma, acral lentiginous melanoma), kidney cancers (e.g., renal cell cancer, adenocarcinoma, hypernephroma, fibrosarcoma, transitional cell cancer (renal pelvis and/or ureter), Wilms' tumor), bladder cancers (e.g., transitional cell carcinoma, squamous cell cancer, adenocarcinoma, and carcinosarcoma). In addition, cancers include myxosarcoma, osteogenic sarcoma, endotheliosarcoma, lymphangio endothelio sarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas.

As used throughout, the term “cell proliferative disorder” also includes rheumatoid arthritis and other auto-immune disorders that are often characterized by inappropriate proliferation of cells of the immune system.

As used throughout, a subject can be a vertebrate, more specifically a mammal (e.g., a human, horse, cat, dog, cow, pig, sheep, goat, mouse, rabbit, rat, and guinea pig). The term does not denote a particular age or sex. Thus, adult, newborn and pediatric subjects, whether male or female, are intended to be covered. As used herein, patient or subject may be used interchangeably and can refer to a subject with or at risk of developing a disorder. The term patient or subject includes human and veterinary subjects. In any of the methods provided herein, the subject can be a subject diagnosed with cancer, an infection or an autoimmune disease.

Any of the methods provided herein can further comprise administering a second therapeutic agent to the subject. The second therapeutic agent can be selected from the group consisting of a chemotherapeutic agent, an adjuvant, an immunomodulatory agent, a vaccine, a tumor antigen, or a combination thereof. In some instances, the second therapeutic agent is a prodrug that can be converted into a toxic drug by a prodrug activator encoded by the retroviral system. In cases, where the second therapeutic is a nucleic acid sequence encoding a therapeutic polypeptide, the second therapeutic agent can be delivered by viral or non-viral means.

Representative chemotherapeutic agents include, but are not limited to amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epirubicin, etoposide, fludarabine, fluorouracil, gemcitabine, hydroxycarbamide, idarubicin, ifosfamide, irinotecan, leucovorin, liposomal doxorubicin, liposomal daunorubicin, lomustine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, tegafur-uracil, temozolomide, teniposide, thiotepa, tioguanine, topotecan, treosulfan, vinblastine, vincristine, vindesine, vinorelbine, or a combination thereof. Representative pro-apoptotic agents include, but are not limited to fludarabinetaurosporine, cycloheximide, actinomycin D, lactosylceramide, 15d-PGJ(2) and combinations thereof.

It is understood that combinations, for example, a composition comprising one or more of the viral vectors described herein and a second therapeutic agent can be administered either concomitantly (e.g., as an admixture), separately but simultaneously (e.g., via separate intravenous lines into the same subject), or sequentially (e.g., one of the compositions or agents is given first followed by the second). Any of the methods provided herein can further comprise radiation therapy or surgery.

As used herein, the term “therapeutically effective amount” or “effective amount” refers to an amount of a composition that, when administered to a subject, is effective, alone or in combination with additional agents, to treat a disease or disorder either by one dose or over the course of multiple doses. A suitable dose can depend on a variety of factors including the particular composition or system used and whether it is used concomitantly with other therapeutic agents. Other factors affecting the dose administered to the subject include. e.g., the type or severity of the disease. For example, a subject having pancreatic cancer may require administration of a different dosage than a subject with brain cancer.

The effective amount of a compound (for example, a chemotherapeutic agent or an immunomodulator) described herein or pharmaceutically acceptable salts or prodrugs thereof can be determined by one of ordinary skill in the art and includes exemplary dosage amounts for a mammal of from about 0.5 to about 200 mg/kg of body weight of active compound per day, which can be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about mg/kg of body weight of active compound per day. Other factors that influence dosage can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject also depends upon the judgment of the treating medical practitioner.

When administering viral vectors (i.e., recombinant vector plasmids, recombinant vector virions, infectious viral particles, or recombinant vector particles), an effective amount of any of the viral vectors described herein will vary and can be determined by one of skill in the art through experimentation and/or clinical trials. For example, for in vivo injection, an effective dose can be from about 10⁶ to about 10¹⁵ recombinant vectors or recombinant vector virions. For example, about 10⁶, 10⁷, 10⁸, 10⁹, 10¹⁰, 10¹¹, 10¹², 10¹³, 10¹⁴, 10¹³ recombinant vectors or recombinant vector virions (e.g., virus particles) or any amount in between these amounts can be administered. In another example, about 10⁶ to about 10⁷, about 10⁶ to about 10⁸, about 10⁶ to about 10⁹, about 10⁶ to about 10¹⁰, about 10⁶ to about 10¹¹, about 10⁶ to about 10¹², about 10⁶ to about 10¹³, or about 10⁶ to about 10¹⁴ recombinant vectors or recombinant vector virions are administered. Effective doses for any of the administration methods described herein can be extrapolated from dose-response curves derived from in vitro or animal model test systems.

As used herein, administer or administration refers to the act of introducing, injecting or otherwise physically delivering a substance as it exists outside the body (e.g. a retroviral system described herein) into a subject, such as by mucosal, intradermal, intravenous, intratumoral, intramuscular, intrarectal, oral, subcutaneous delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof.

The recombinant viral systems. e.g., retroviral systems, are administered via any of several routes of administration, including orally, parenterally, intramucosally, intravenously, intratumorally, intraperitoneally, intraventricularly, intramuscularly, subcutaneously, intracranially, intracavity or transdermally. Administration can be achieved by, e.g., topical administration, local infusion, injection, or by means of an implant.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXEMPLARY EMBODIMENTS

Exemplary embodiments of the invention include:
1. A recombinant virus system comprising: (a) a first virus (i) encoding a first regulatory element operably linked to a nucleic acid encoding a first activator; and (ii) lacking a coding sequence for at least one viral protein required for replication such that the first virus is a replication-deficient virus; and (b) a second virus comprising a nucleic acid comprising a first polynucleotide encoding the viral protein or proteins necessary for viral replication that are lacking in the first virus, wherein the first polynucleotide is only expressed when the first activator activates expression of the first polynucleotide and/or its encoded viral protein(s).
2. The recombinant virus system of embodiment 1, wherein the first and the second viruses are retroviruses or adenoviruses.
3. The recombinant virus system of embodiment 1 or 2, wherein the activator activates expression by increasing transcription or translation of the first polynucleotide.
4. The recombinant virus system of any one of embodiments 1-3 wherein the second virus comprises a second regulatory element operably linked to the first polynucleotide encoding a viral protein necessary for viral replication, and wherein the first activator activates transcription of the first polynucleotide by binding to the second regulatory element.
5. The recombinant virus system of any one of embodiments 1-4, wherein the activator binds to the second regulatory element to activate transcription, wherein the second regulatory element is a promoter, an enhancer or a repressor binding sequence.
6. The recombinant virus system of embodiment 1, wherein the first activator is a de-repressor; and the first polynucleotide sequence encoded by the second retrovirus is only expressed when the de-repressor activates expression by de-repressing expression of the first polynucleotide and/or viral protein(s).
7. The recombinant virus system of embodiment 6, wherein de-repression occurs at the transcriptional level or the translational level.
8. The recombinant virus system of any one of embodiments 1-7, wherein the first regulatory element and/or the second regulatory element is selected from the group consisting of a promoter, an enhancer, a promoter/enhancer combination, an internal ribosome entry site, an epigenetic regulator and a translation regulator.
9. The recombinant virus system of embodiment 8, wherein the promoter is a constitutive or an inducible promoter.
10. The recombinant virus system of any one of embodiments 1-9, wherein the second virus is a replicating virus encoding all viral proteins necessary for viral replication.
11. The recombinant virus system of any one of embodiments 1-10, wherein the first and/or second virus further comprises a heterologous expression cassette comprising a payload promoter operably linked to a payload polynucleotide sequence.
12. The recombinant virus system of any one of embodiments 1-11, wherein the viral protein required for replication is selected from the group consisting of gag, env, pol, rev and tat.
13. The recombinant virus system of any one of embodiments 2-12, wherein the retroviruses are selected from the group consisting of lentivirus, murine leukemia virus (MLV), Moloney murine leukemia virus (MoMLV), foamy virus.
14. The recombinant virus system of any one of embodiments 11-13, wherein the payload polynucleotide encodes a polypeptide selected from the group consisting of a therapeutic protein, a prodrug activator, a cytotoxic protein, and a reporter protein.
15. The recombinant virus system of embodiment 14, wherein the prodrug activator is thymidine kinase, cytidine deaminase or purine nucleoside phosphorylase (PNP).
16. The recombinant virus system of any one of embodiments 1-15, wherein the first activator is selected from the group consisting of HIV-1 trans-activator protein (Tat), HIV-1 Rev, Gal4-VP16, GAL4FF, GAL4-VP64, and VP16-E2 and tetracycline transactivator protein.
17. The recombinant virus system of embodiment 1 or 2, wherein the second virus is a replication-deficient virus and encodes a third regulatory element operably linked to a nucleic acid encoding a second activator; and the first virus comprises a nucleic acid comprising a second polynucleotide encoding a viral protein necessary for viral replication, wherein the second polynucleotide is only expressed when the second activator activates expression of the second polynucleotide, and wherein the first and second viruses can only replicate when the first and second activators are expressed.
18. The recombinant virus system of embodiment 17, wherein the first virus comprises a fourth regulatory element operably linked to the second polynucleotide encoding a viral protein necessary for viral replication, and wherein the first activator activates transcription of the second polynucleotide by binding to the fourth regulatory element.
19. The recombinant virus system of embodiment 17 or 18, wherein the third regulatory element or the fourth regulatory element is selected from the group consisting of a promoter, an enhancer, a promoter/enhancer combination, an internal ribosome entry site, an epigenetic regulator and a translational regulator.
20. The recombinant virus system of embodiment 19, wherein the promoter is a constitutive or an inducible promoter.
21. The recombinant virus system of any of embodiments 18-20, wherein the first virus or second virus, or both, comprise a heterologous expression cassette comprising a payload promoter operably linked to a payload polynucleotide.
22. The recombinant virus system of embodiment 21, wherein the payload polynucleotide encodes a polypeptide selected from the group consisting of a therapeutic protein, a prodrug activator, a cytotoxic protein, and a reporter protein.
23. The recombinant virus system of any one of embodiments 17-22, wherein the viral protein required for replication is selected from the group consisting of gag, env, pol, rev and tat.
24. The recombinant virus system of embodiments 17-23, wherein the retroviruses are selected from the group consisting of lentivirus, murine leukemia virus (MLV), Moloney murine leukemia virus (MoMLV), and foamy virus.
25. The recombinant virus system of embodiments 17-24, wherein the payload polynucleotide encodes a polypeptide selected from the group consisting of a therapeutic protein, a prodrug activator, a cytotoxic protein, and a reporter protein.
26. The recombinant virus system of embodiment 25, wherein the prodrug activator is thymidine kinase, cytidine deaminase or purine nucleoside phosphorylase (PNP).
27. The recombinant virus system of any one of embodiments 17-26, wherein the third or fourth regulatory element is selected from the group consisting of a constitutive promoter, an inducible promoter, and a tissue-specific promoter.
28. The recombinant virus system of any one of embodiments 17-27, wherein the activator is selected from the group consisting of HIV-1 trans-activator protein (Tat), HIV-1 Rev, Gal4-VP16, and VP16-E2 and tetracycline transactivator protein.
29. The recombinant virus system of any one of embodiments 17-28, wherein the activator is selected from the group consisting of HIV-1 trans-activator protein (Tat), HIV-1 Rev, Gal4-VP16, and VP16-E2 and tetracycline transactivator protein.
30. The recombinant virus system of any one of embodiments 17-29, wherein one or more LTRs (e.g., 3′LTR and./or 5′LTR) of the first and/or second vector comprises a deletion, optionally, in the U3 region of the LTR.
31. The recombinant virus system of any one of embodiments 17-30, wherein the one or more LTRS of the first and/or second vector comprises an insertion, optionally wherein the insertion is a promoter (e.g., a CMV promoter), and optionally wherein the insertion is one or more binding sites for an activator, optionally wherein the binding sites are GAL4 binding sites.
32. A method for making a recombinant virus system comprising: (a) transfecting a first suitable host cell with the first viral vector of any of embodiments 1-31; (b) transfecting a second suitable host cell with the second viral vector of any of embodiments 1-31; and (c) recovering the first and second retroviruses.
33. A method for transfecting a target cell with a replicating virus system comprising contacting the target cell with the first virus and the second virus of any one of embodiments 1-31.
34. The method of embodiment 33, wherein the cell is a mammalian cell.
35. The method of any of embodiments 32-34, wherein the cell is contacted in vitro, ex vivo or in vivo.
36. A pharmaceutical composition comprising: (a) the first virus and/or the second virus of the system of any of embodiments 1-31; and (b) and a pharmaceutical carrier.
37. A method of treating a disease in a subject in need thereof comprising administering the system of any one of embodiments 1-31, or the pharmaceutical composition of embodiment 36, to the subject.
38. The method of embodiment 37, wherein the disease is a cell proliferative disorder.
39. The method of embodiment 38, wherein the cell proliferative disorder is selected from the group consisting of lung cancer, breast cancer, ovarian cancer, uterine cancer, prostate cancer, testicular cancer, kidney cancer, urinary tract cancer, oral cancer, head and neck cancer, esophageal cancer, gastric cancer, pancreatic cancer, colorectal cancer, skin cancer, melanoma, sarcoma, lymphoma, leukemia, and brain cancer including glioblastoma, anaplastic astrocytoma, oligodendroglioma, medulloblastoma.
40. The method of embodiment 39, wherein the cancer is glioblastoma.
41. The method of any one of embodiments 37-40, wherein the first or second polynucleotide encodes a prodrug activator, and wherein the method further comprises administering a prodrug to the subject, such that when the prodrug activator is expressed, the prodrug activator converts the prodrug into a toxic drug.
42. The method of any one of embodiments 37-41, wherein the first and/or second viral vector is administered to the subject as a plasmid or as an infectious retroviral particle.
43. The method of any one of embodiments 37-42, wherein the subject is a mammal.
44. The method of embodiment 43, wherein the subject is a human.
45. The method of any of embodiments 3744, wherein administration is systemic, topical or local administration.
46. The method of any one of embodiments 37-45, wherein the first and the second virus of the system are administered simultaneously or sequentially to the subject.

### Example I

Construction of pLXV-GAL4, pAC3-Minimal and pAC3-Maximal Plasmids

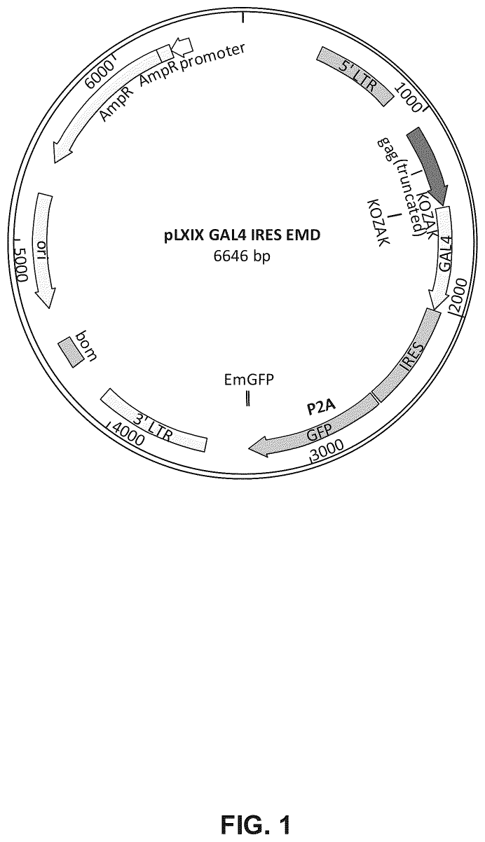

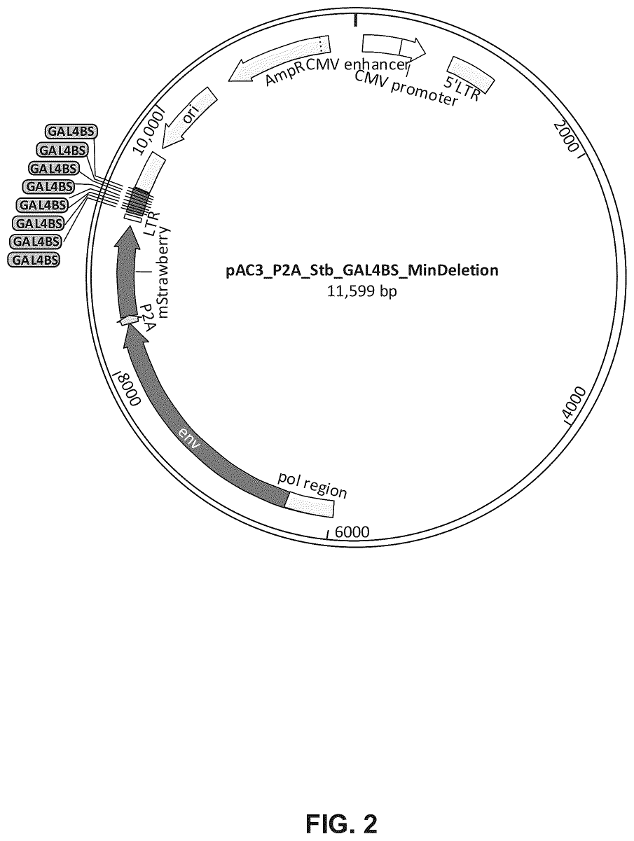

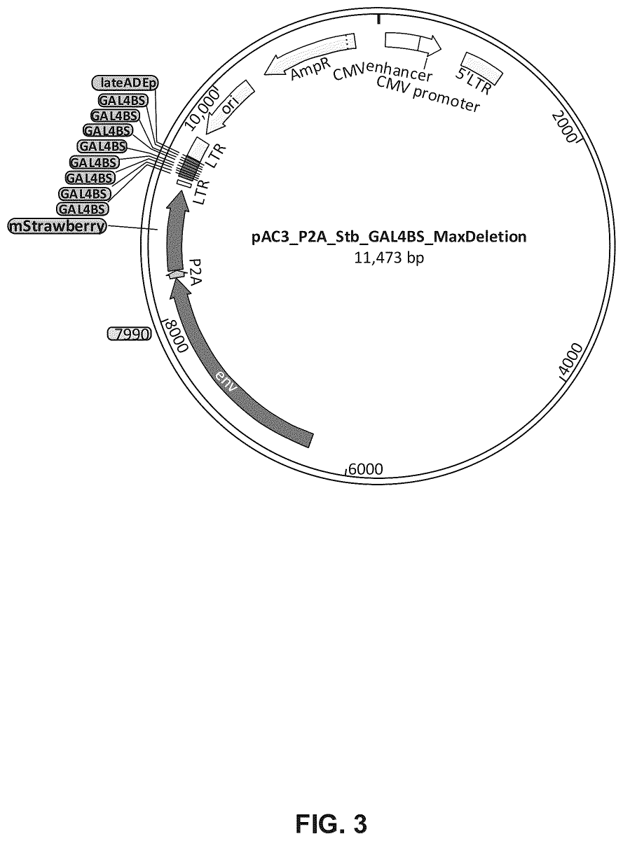

Gibson assemble cloning was utilized to place a codon and stability optimized genetic sequence for the GALFFF (i.e., a GAL4/VP16 fusion protein comprising GAL fused with parts of VP16) into pLXSN (Takara Bio Inc., Shiga, Japan) followed by an internal ribosome entry site (FIG. 1). pAC3-Minimal and pAC3-Maximal were also created using Gibson assembly cloning. In these plasmids the U3 region of the MMLV 5′ LTR is replaced with the CMV promoter to drive the initial round of gene expression upon transfection. Deletions were made in the U3 of the native MMLV 3′ LTR to disrupt native promoter function. During the process of reverse transcription the U3 region of the 3′ LTR is transposed to the U3 region of the 5′ LTR in the proviral DNA. This results in an infectious vector with identical modified LTR sequences at the 5 and 3′ ends. pAC3-Minimal (FIG. 2) included a deletion in the native enhancer sequence. pAC3-Maximal (FIG. 3) included a deletion in the enhancer sequence and viral CCAAT box. Deleted sequences were replaced with GAL4 binding sites. Strawberry and Emerald fluorescent proteins were placed into viral constructs downstream of either a P2A or internal ribosome entry site, respectively.

Construction of pLXIX-Tat and pAC3-TIN Plasmids.

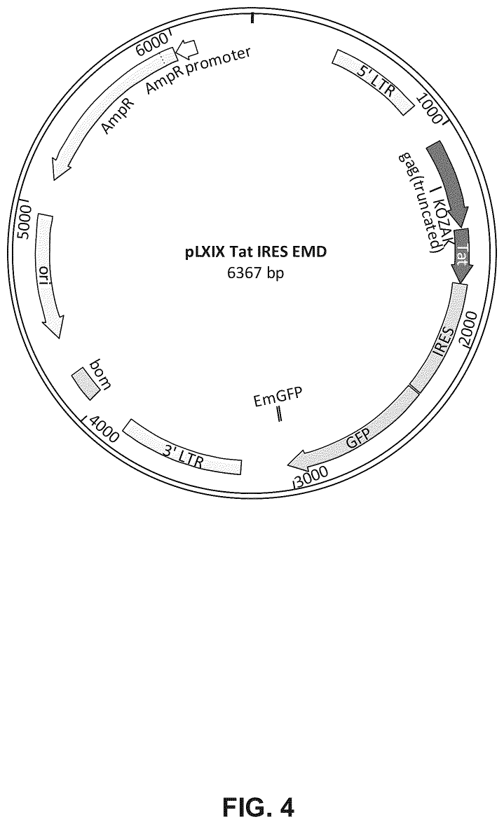

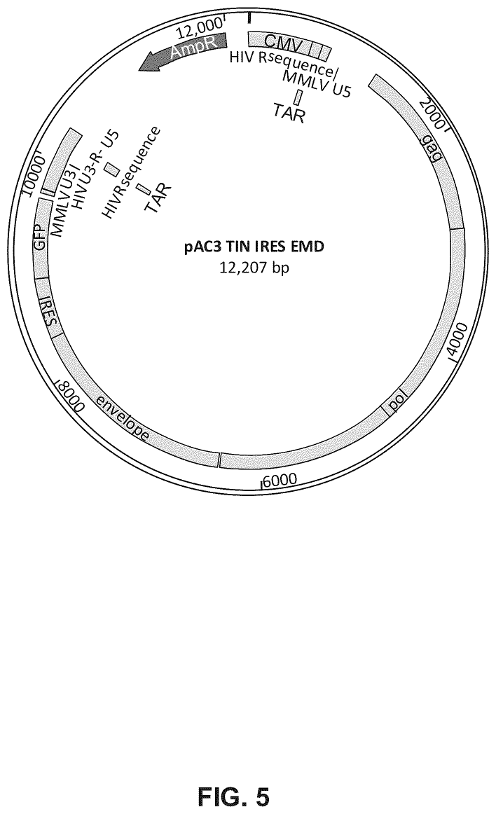

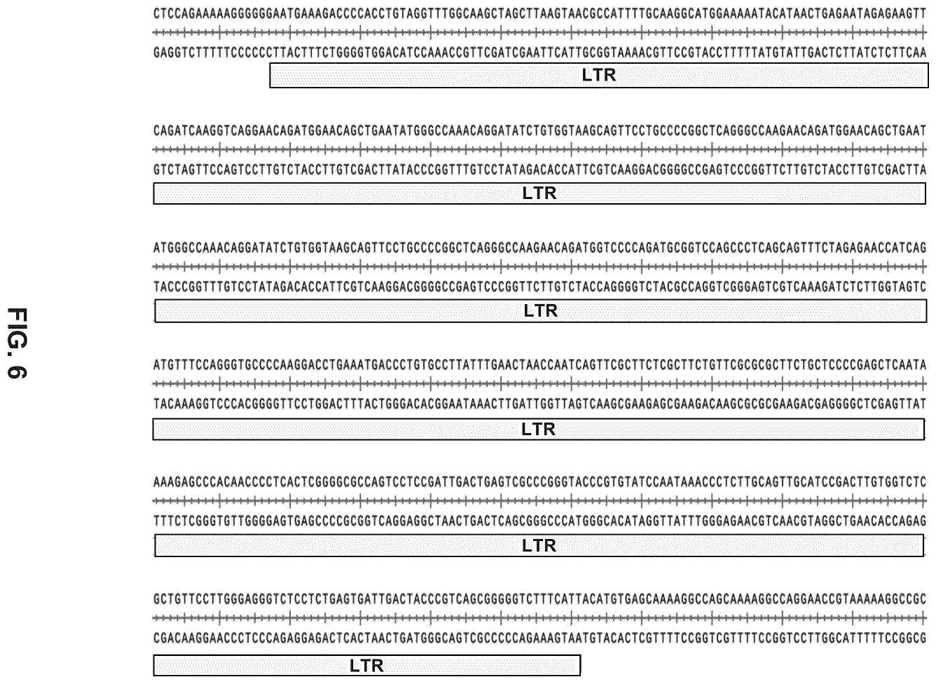

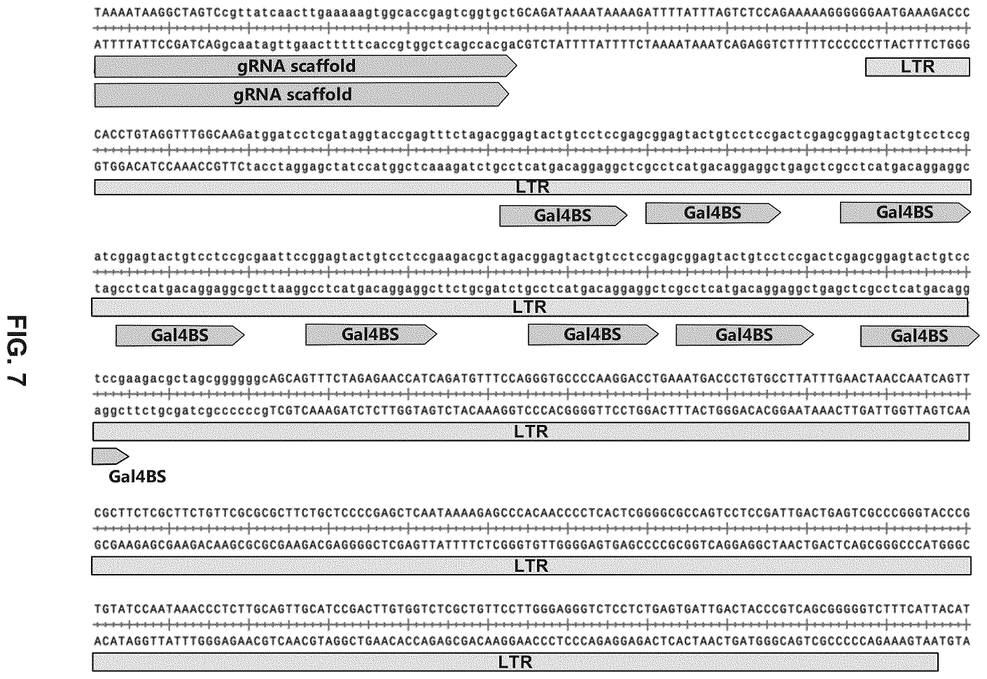

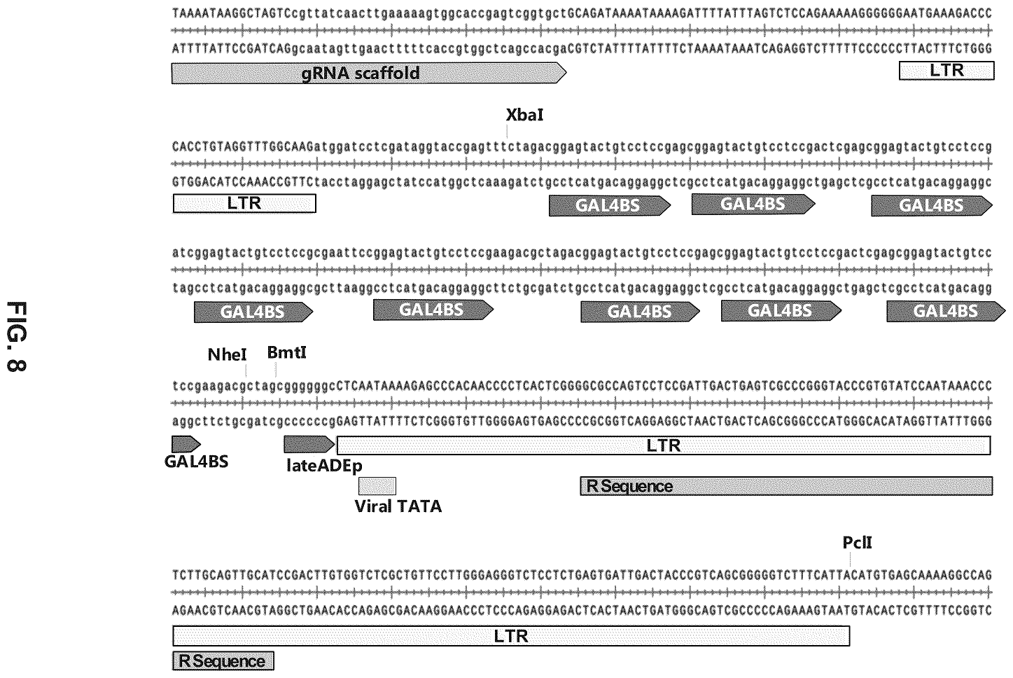

Gibson assembly was utilized to place a codon and stability optimized genetic sequence for the HIV-1 Tat sequence into pLXSN (Takara Bio Inc.) followed by an internal ribosome entry site (FIG. 4). pAC3-TIN was created by replacing the MMLV 3′ LTR with the HIV-1 U3, R, and U5 sequences from the pHR plasmid (Addgene) (FIG. 5). The 5′ LTR native MMLV R sequence was also replaced with the HIV-1 R sequence.

### Cell Lines and Culture

Human embryonic kidney 293T cells, with stable gag-pol expression (Retro-X cells, purchased from Takara, Inc) were cultured in Dulbecco's modified Eagle's medium-nutrient mixture supplemented with 10% fetal bovine serum and 1× Gibco GlutaMAX (Gibco, Inc.). Murine glioblastoma SB28 (provided by Dr. Hideho Okada, University of California San Francisco, San Francisco, CA) were cultured in RPMI 1640 medium, supplemented with 10% fetal bovine serum, Gibco GlutaMAX (Gibco, Inc., Waltham, MA), non-essential amino acids (Gibco, Inc.), hydroxyethyl piperazineethanesulfonic acid (Gibeo, Inc.), Penicillin-Streptomycin (Gibco, Inc.), and 0.1% beta-mercaptoethanol.

### Viral Production and Concentration

Virus was produced via transient transfection of Retro-X producer cells. Reverse transfection utilizing Fugene HD (Promega) and 10 micrograms of viral plasmid DNA and plasmid DNA containing the VSV-G envelope was implemented. Virus containing supernatant was collected at approximately 36-48 hours after initial transfection. For in vivo studies virus was concentrated using column-based retrovirus purification with buffer exchange to phosphate buffer solution (PBS) (Bioland Scientific LLC.). Functional viral titers in transducing units per mL (TU/mL) were determined via flow-cytometry.

### In Vitro Viral Replication and Stability

In brief, specific multiplicities of infection (MOI) were added to SB28 tumor cells in vivo and allowed to replicate over time, with transduction levels measured at regular intervals via flow cytometry for fluorescent protein transgenes. Azidothymidine, which inhibits viral spread, was utilized for control groups. An MOI of 0.3 was commonly utilized for in vitro viral spread experiments.

### Mice

8-12 week old C57BL/6 mice were purchased from Jackson Laboratories and maintained at the University of California San Francisco.

### Animal Studies.

SB28 murine glioblastoma tumor cells were used for in vivo experiments. All cells stably expressed luciferase to allow for bioluminescent imaging and also stably expressed LSSmOrange, allowing for identification of the tumor cells on flow cytometry. On day 0, 10,000 tumor cells were implanted intracranially. Cells were implanted using a stereotactic frame at the following coordinates from the bregma: anteroposterior (AP), 0 mm; mediolateral (ML), 1.9 mm; and dorso-ventral (DV), 3.0 mm. On day 4 post-tumor implantation, mice were injected with 5×10³ TU of pLXIX-GAL4-EMD and pAC-Minimal-Strawberry or pLXIX-GAL4-EMD and pAC-Maximal-Strawberry. Pre-mix experiments using pre-transduced co-infected positive cells mixed with uninfected tumor cells to a total of 4% co-infected cells and 96% uninfected tumor cells were also performed.

### Analysis of Viral Spread

Mice were sacrificed at day 4, 15, and 18 post-tumor injection time points. Day 15 and 18 time points were combined during analysis. Brain tumors were minced and placed into collagenase type IV (Thermo Fisher Scientific #17104019) and Deoxyribonuclease I (Worthington Biochemical Corporation) solutions for processing while shaking at 37 C. Tumors were then filtered through 70 um filters, and red blood cells were lysed using Ammonium-Chloride-Potassium (ACK) lysing buffer (Lonza). Flow cytometric analysis was then performed. Acquisition was conducted on an Attune NxT Flow Cytometer (Thermo Fisher Scientific, Waltham, MA). Analysis of flow cytometry results was performed using FlowJo software.

### Results

### Minimal and Maximal Insertions of GAL4 Binding Sites Differentially Modify Replicating Retrovirus Replication and Viral Transgene Expression

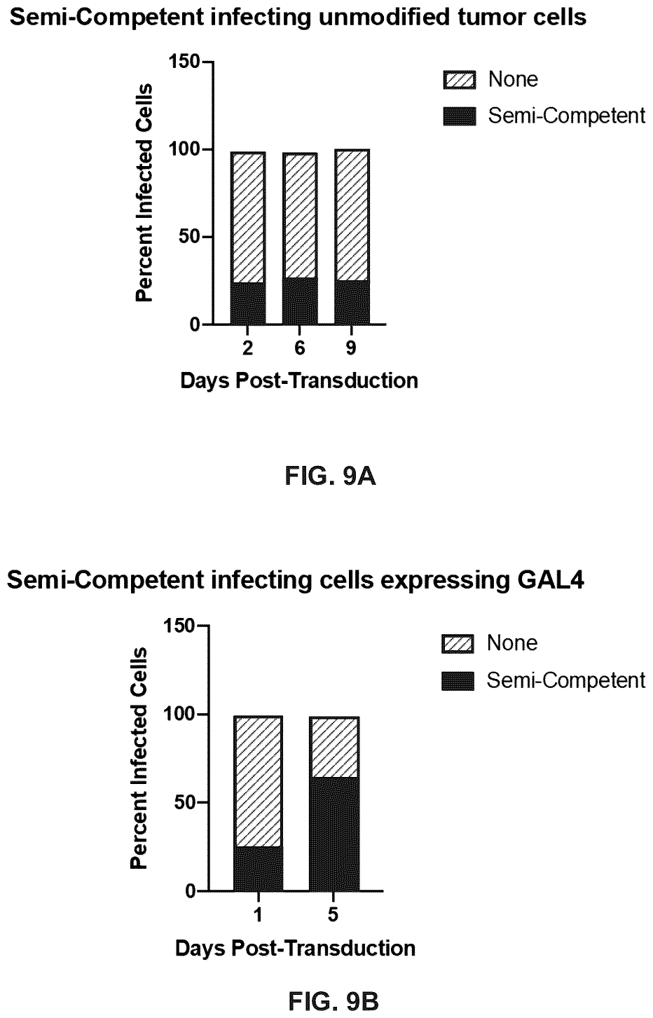

Insertion/deletions were made in the U3 of the 3′ MMLV LTR, to differentially modify viral replication and expression, providing two replicating viruses (RRVs) with varying amounts of dependence on GAL4/VP16 expression. An exemplary, unmodified 3′ MMLV LTR sequence for the pAC3-Strawberry vector is provided in FIG. 6 (SEQ ID NO: 1). This 3′ MMLV LTR sequence was modified by making deletions in the U3 of the native MMLV 3′ LTR to disrupt native promoter function. pAC3-Minimal (FIG. 2) included a deletion in the native enhancer sequence. pAC3-Maximal (FIG. 3) included a deletion in the enhancer sequence and viral CCAAT box. Deleted sequences were replaced with GAL4 binding sites as shown in FIGS. 7 and 8. FIGS. 7 and 8 provide the 3′ MMLV LTR sequences, SEQ ID NO: 2, and SEQ ID NO: 3, respectively, used in the minimal deletion and maximum deletion vectors, respectively, described herein. By varying the number of GALA binding sites in the vector, viral replication and expression of the binary vectors of the systems described herein can be modulated. When applied to SB28 tumor cells, a strawberry transgene carrying RRV with a minimal deletion (pAC-Minimal-Strawberry) demonstrated robust expression, but no viral replication over nine days in culture (FIG. 9A). An RRV with the maximal deletion (pAC-Maximal-Strawberry) demonstrated no expression at all in a similarly performed experiment. Stable expression of GAL4/VP16 in the SB28 tumor cells permitted replication of the pAC-Minimal-Strawberry virus (FIG. 9B).

In Vitro Addition of a GAL4/VP16 Carrying Defective Retrovirus Permits Expression and Replication of Replicating Retroviruses with Minimal and Maximal Insertions of GAL4 Binding Sites

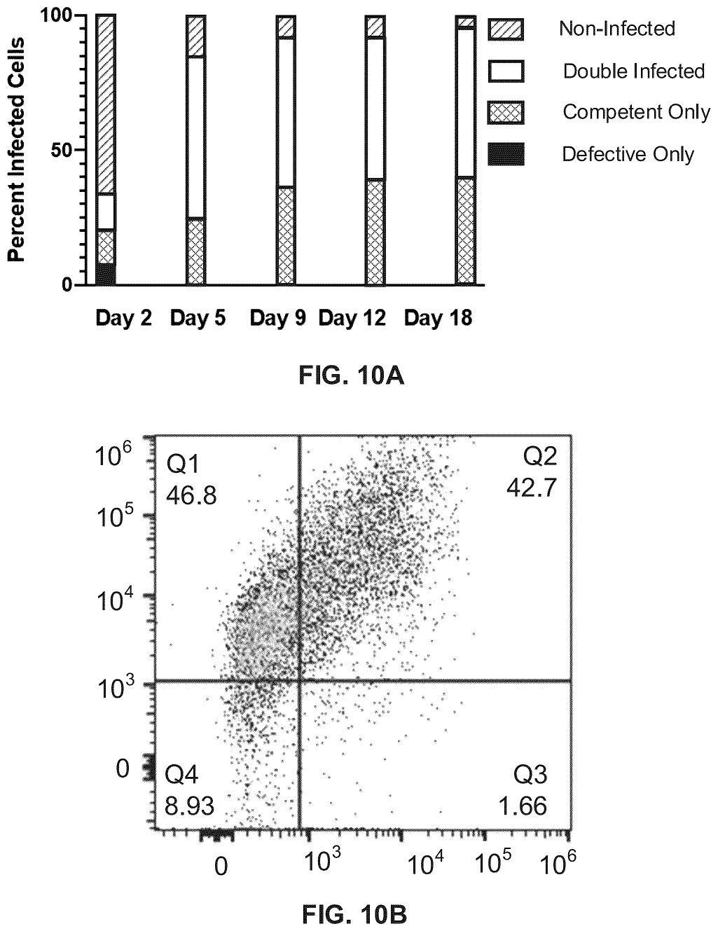

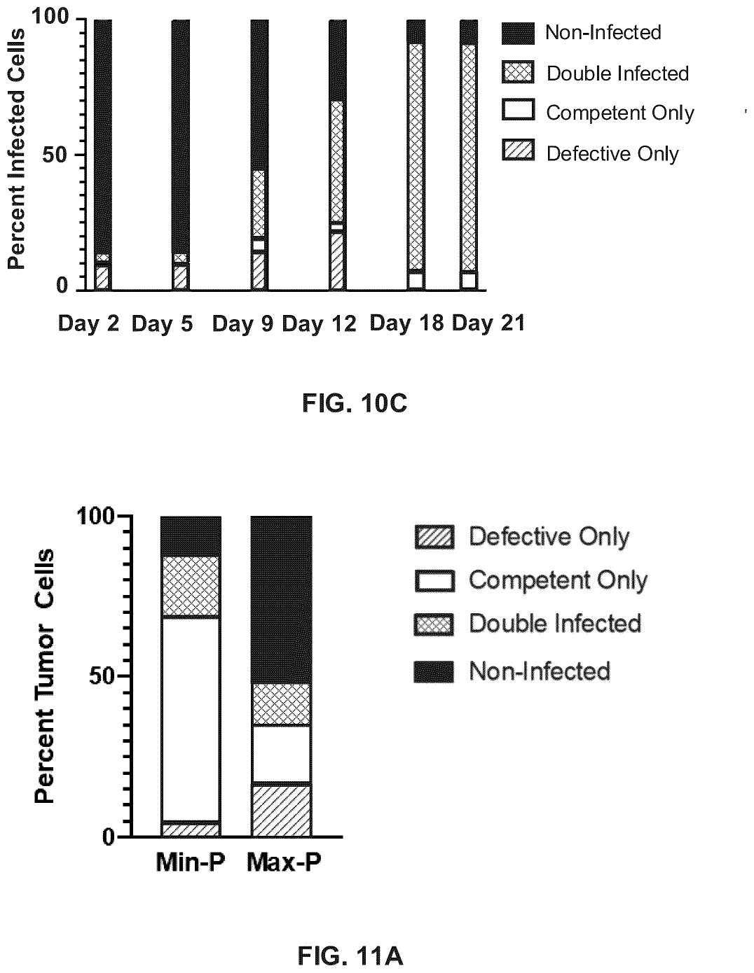

After confirming the lack of replication in pAC-Minimal-Strawberry and pAC-Maximal-Strawberry without concurrent GAL4/VP16 expression, an experiment to assess the ability for GAL4 to permit viral replication was performed. First, pAC-Minimal-Strawberry and pLXIX-GAL4-EMD were added to SB28 cells at an MOI of 0.3 per virus. Viral spread was then monitored via flow cytometry for EMD and Strawberry at regularly spaced time points. In contrast to pAC-Minimal-Strawberry alone, the addition of pLXIX-GAL4-EMD and pAC-Minimal-Strawberry together to SB28 glioblastoma tumor cells allowed for robust viral replication and spreading of both viruses (FIG. 10A). In addition, co-infected cells demonstrated higher expression of Strawberry expression, highlighting the synergistic nature of the binary replication system (FIG. 10B). A similar experiment was then conducted with the pAC-Maximal-Strawberry. Similarly, the addition of pLXIX-GAL4-EMD and pAC-Maximal-Strawberry together, to SB28 glioblastoma tumor cells, allowed for robust viral replication and spreading of both viruses to an extremely high percentage of double positive cells (FIG. 10C).

### In Vivo Assessments of the Minimal and Maximal Binary Systems Demonstrate Robust Co-Infection and Replication

Following the success of in vitro experiments for both the Minimal and Maximal Binary systems, the two systems were tested in an intracranial mouse tumor. In one set of experiments, the systems were assessed via implantation of pre-mixed tumors (4% co-infected cells and 96% uninfected tumor). Pre-mix experiments demonstrated robust replication and expansions of a co-infected tumor cell population (FIGS. 11A-D). Similar in vivo experiments performed using injection of the viruses at low viral titers, to challenge the system, also demonstrated excellent replication and a substantial co-infected tumor cell population (FIGS. 12A-C). No fluorescent protein expression was detected 4 days after viral injection, further demonstrating the robust spread and replicative capacity of the system.

### Example II

### Regulated Retroviral Vectors for Gene Editing

As described below, a vector system comprising a defective virus encoding Cas9 and a replication competent virus encoding a cognate guide RNA (gRNA) for a gene of interest can be used to edit the genome of the cell. This system may be used to knock out any gene for which a functional gRNA is present in tumor cells.

### Construction of Plasmids

Gibson assembly cloning was utilized to place the EGFP-T2A-Cas9 from pRubiG-T2A-Cas9 (Williams et al., Sci Rep 2016, 6, 25611, doi:10.1038/srep2561) (Addgene plasmid #75348; http://n2t.net/addgene:75348: RRID:Addgene_75348) into pLXIX-GAL4 (described above) immediately downstream of a P2A sequence creating a replication defective retrovirus (pLXIX-Gal4-P2A-EGFP-T2A-Cas9) expressing Gal 4 and Cas9 (FIG. 16). This results in both Cas9 and EGFP expression in infected cells, allowing easy identification and detection.

gRNA Vector

The human β2-microglobulin gene target sequence was used to generate the sgRNA spacer sequence by insertion into two 60mer oligonucleotides as indicated below (sequences are 5′ to 3′, and the regions marked in bold are reverse complements of each other):

B2MsgRNA_F:
(SEQ ID NO: 5)
TTTCTTGGCTTTATATATCTTGTGGAAAGGACGAAACACCGAGTAGCGC
GAGCACAGCTA
B2MsgRNA_R:
(SEQ ID NO: 6)
GACTAGCCTTATTTTAACTTGCTATTTCTAGCTCTAAAACTAGCTGTGC
TCGCGCTACTC

The two oligos were annealed and extended to make a 100 bp double stranded DNA fragment using Phusion flash polymerase (NEB). The gRNA cloning vector (Addgene plasmid #41824; http://n2t.net/addgene:41824; RRID:Addgene_41824) was linearized using AfIII and the 100 bp DNA fragment was incorporated using Gibson assembly (Mali et al.. Science 2013, 339, 823-826, doi:10.1126/science.1232033). The resulting virus was the U6-B2MsgRNA virus (FIG. 17A). This vector served as a template for insertion of the U6-B2MsgRNA expression cassette into the Not1 site of the minimal (FIGS. 17B, and 18A) and maximal deletion RRV vectors (FIGS. 17C, and 18B) using Gibson assembly.

### Cell Lines and Culture

Human embryonic kidney 293T cells (ATCC), were cultured in Dulbecco's modified Eagle's medium-nutrient mixture supplemented with 10% fetal bovine serum and 1% Penicillin/streptomycin (Corning, Inc.). U87EGFRvIII cells were cultured in DMEM medium, supplemented with 10% fetal bovine serum, 1% Penicillin-Streptomycin (Corning, Inc.).

### Viral Production and Concentration

Replication defective (Cas9 encoding vector) was produced via transient calcium phosphate co-transfection of 293T cells with viral plasmid DNA, pHIT60 packaging plasmid, plasmid DNA containing the VSV-G envelope.

Minimal (i.e., small deletion) and maximal (i.e., large deletion) deletion vectors were produced via transient calcium phosphate co-transfection of 293T cells with viral plasmid DNA, and plasmid DNA containing GAL4 gene. Virus containing supernatant was collected at approximately 36-48 hours after initial transfection. For in vitro studies, virus was concentrated using RetroX concentrator (Takara) per manufacturers protocol and resuspended in PBS. Functional viral titers in transducing units per mL (TU/mL) were determined via flow-cytometry.

### In Vitro Viral Replication and Assessment of β2-Microglobulin Knockdown

In brief, specific multiplicities of infection (MOI) were added to U87EGFRvIII tumor cells in vitro and allowed to replicate over time, with transduction levels measured at regular intervals via flow cytometry for fluorescent protein transgenes. MOIs of 0.01-0.1 were commonly utilized for in vitro viral spread experiments. Knockdown of B2M was assessed by cell surface staining of transduced cells. Briefly, cells were harvested and washed twice in staining buffer (PBS supplemented with 0.2% BSA). Up to 1×106 cells/100 μl was aliquoted into FACS tubes. 5 μl of APC conjugated anti-human β2-microglobulin antibody (Biolegend) or isotype control (IgG1κ, Biolegend) was added and cells were incubated at 4° C. for 30 minutes with agitation. Cells were washed twice with PBS containing 0.2% BSA, fixed with 4% PFA for 15 minutes, washed twice more and resuspended in 300 μl of staining buffer for FACS analysis.

### Retrovirus Replication and Knockdown of β2-Microglobulin in U87vIII Cells

pAC-Minimal (i.e. small deletion)-strawberry-U6-B2MsgRNA vector and pLXIX-GAL4-EGFP-CAS9 were added to U87vIII cells at multiplicity of infection (moi) of 0.01 and 0.1, respectively. Virus spread was then monitored via flow cytometry for EMD and strawberry at regularly spaced time points. In contrast to pLXIX-GAL4-EGFP-CAS9 alone, addition of pAC-Minimal-strawberry-U6-B2MsgRNA allowed for viral replication and spread of both viruses (FIG. 19). In cells that had received both the pAC-Minimal-strawberry-U6-B2MsgRNA and pLXIX-GAL4-EGFP-CAS9, β2-microglobulin knockdown was also assessed by cell surface antibody staining. Up to 50% of cells cotransduced (ie GFP and strawberry positive) showed no APC/β2M positivity by day 20 post transduction (FIG. 20).

Similar experiments can be conducted with the pAC-Maximal (i.e. large deletion)-strawberry-U6-B2MsgRNA vector and pLXIX-GA L4-EGFP-CAS9 by adding these vectors to U87vIII cells at multiplicity of infection (moi) of 0.01 and 0.1, respectively. Virus spread can be monitored via flow cytometry for EMD and strawberry at regularly spaced time points. It is expected that, in contrast to pLXIX-GAL4-EGFP-CAS9 alone, addition of pAC-Maximal strawberry-U6-B2MsgRNA will allow for viral replication and spread of both viruses. In cells that receive both the pAC-Maximal-strawberry-U6-B2MsgRNA and pLXIX-GAL4-EGFP-CAS9, β2-microglobulin knockdown can also assessed by cell surface antibody staining. It is expected that infection with pAC-Maximal-strawberry-U6-B2MsgRNA and pLXIX-GAL4-EGFP-CAS9 will result in greater than 85% cells co-infected. Analysis of cell surface B2M expression in the total cell population, and in the co-transduced population, as described above should result in knockdown of B2M in greater than 50% of U87vIII cells.

### Inhibition of Tumor Growth in a Glioblastoma Animal Model

The human tumor specific EGFR deletion variant, EGFRvIII target sequence, will be used to generate the sgRNA spacer sequence by insertion into two 60mer oligonucleotides as indicated below (sequences are 5′ to 3′, and the regions marked in bold are reverse complements of each other):

EGFRvIIIgRNA_F:
(SEQ ID NO: 12)
TTTCTTGGCTTTATATATCTTGTGGAAAGGACGAAACACCGGAAAAGAA
AGGTAATTATG
EGFRvIIIgRNA_R:
(SEQ ID NO: 13)
GACTAGCCTTATTTTAACTTGCTATTTCTAGCTCTAAAACCATAATTAC
CTTTCTTTTCC

These oligos will be used to generate the U6-EGFRvIIIgRNA expression vector, to be used as a template for generating the minimal and maximal deletion U6-EGFRvIIIgRNA vectors as described previously.

These minimal or maximal deletion vectors carrying both the EGFRvIIIgRNA and the strawberry fluorescent marker gene will be co-administered with pLXIX-GAL4-EGFP-CAS9 to U87vIII cells in vitro at specific multiplicities of infection. Levels of spread and cotransduction will be assessed as described herein and the effect on EGFRvIII expression will also be assessed. It is expected that co-transduced U87EGFRvIII cells will show spread and co-transduction levels similar to those seen with previous iterations of the vectors. It is also expected that co-transduction will result in a significant reduction in EGFRvIII expression using flow cytometry for the detection of cell surface expression of EGFRvIII, or by western blot to assay EGFRvIII protein levels in cell lysates.

1e⁵ U87vIII human glioma cells, stable for the firefly luciferase gene, will be surgically implanted into athymic nude mice by stereotactic injection. The minimal or maximal deletion vectors carrying both the EGFRvIIIgRNA and the strawberry fluorescent marker gene will be co-administered with pLXIX-GAL4-EGFP-CAS9 via intratumoral stereotactic injection. The vectors will be allowed to spread and tumor growth will be monitored by bioluminescent imaging of the tumors at weekly intervals. It is expected that there will be reduced bioluminescent signals in the CRISPR/CAS9 treated animals compared to untreated control animals over time indicating significant tumor growth inhibition.

### Example III

### Methods

Construction of pLXIX-GAL4-IM and pAC3-GAL4BS-RLI

Gibson assemble cloning was utilized to place a codon and stability optimized genetic sequence for the following genes: IL-7, FLT3L, 4-1BBL into the pLXIX-GAL4 plasmid to create pLXIX-GAL4-IM (FIG. 21A). The IL-15 superagonist RLI was similarly cloned into the pAC-Maximal-Strawberry vector (replacing Strawberry) to create pAC3-GAL4BS-RLI (also referred to as GAL4BS-IL-15)(FIG. 21B).

### Cell Lines and Culture

Human embryonic kidney 293T with stable gag-pol expression (Retro-X cells purchased from Takara. Inc) were cultured in Dulbecco's modified Eagle's medium-nutrient mixture supplemented with 10% fetal bovine serum and IX Gibco GlutaMAX (Gibco, Inc.). Murine glioblastoma SB28 (generously provided by Dr. Hideho Okada, University of California San Francisco. San Francisco, CA) were cultured in RPMI 1640 medium supplemented with 10% fetal bovine serum, Gibco GlutaMAX (Gibco, Inc.), non-essential amino acids (Gibco, Inc.), hydroxyethyl piperazineethanesulfonic acid (Gibco, Inc.), Penicillin-Streptomycin (Gibco, Inc.), and 0.1% beta-mercaptoethanol.

### Viral Production and Concentration

Virus was produced via transient transfection of Retro-X producer cells. Reverse transfection utilizing Fugene HD (Promega) and 10 micrograms of viral plasmid DNA and plasmid DNA containing the VSV-G envelope was implemented. Virus containing supernatant was collected at approximately 36-48 hours after initial transfection. For in vivo studies virus was concentrated using column-based retrovirus purification with buffer exchange to phosphate buffer solution (PBS) (Bioland Scientific LLC.). Functional viral titers in transducing units per mL (TU/mL) were determined via flow-cytometry.

### In Vitro Expression of Therapeutic Transgenes

The plasmids pLXIX-GAL4-IM and pAC3-GAL4BS-RLI were added to SB28 cells in culture at an MOI of 0.3. At 96 hours after infection, cell culture supernatant was collected and enzyme-linked immunosorbent assays (ELISAs) were performed to assess the levels of RLI, IL-7, and FLT3L. Flow cytometry with an anti-4-1BBL antibody was similarly performed to confirm 4-1BBL expression on virus infected cells.

### Mice

8-12 week old C57BL/6 mice were purchased from Jackson Laboratories and maintained at the University of California San Francisco.

### Animal Studies—Viral Spread

SB28 murine glioblastoma tumor cells were used for in vivo experiments. All cells stably expressed luciferase to allow for bioluminescent imaging and also stably expressed LSSmOrange, allowing for identification of the tumor cells on flow cytometry. On day 0, 10,000 tumor cells were implanted intracranially. Cells were implanted using a stereotactic frame at the following coordinates from the bregma: anteroposterior (AP), 0 mm; mediolateral (ML), 1.9 mm; and dorso-ventral (DV), 3.0 mm. On day 4 post-tumor implantation, mice were injected with 5×103 TU of pLXIX-GAL4-EMD and pAC-Minimal-Strawberry or pLXIX-GAL4-EMD and pAC-Maximal-Strawberry. Pre-mix experiments using pre-transduced co-infected positive cells mixed with uninfected tumor cells to a total of 4% co-infected cells and 96% uninfected tumor cells were also performed.

### Animal Studies—Therapeutic Testing

Similarly to above, SB28 murine glioblastoma tumor cells were used for in vivo experiments testing the therapeutic efficacy of virally expressed immune genes. All SB28 cells stably expressed luciferase to allow for bioluminescent imaging. On day 0, 10.000 tumor cells were implanted intracranially. Cells were implanted using a stereotactic frame at the following coordinates from the bregma: anteroposterior (AP), 0 mm; mediolateral (ML), 1.9 mm; and dorso-ventral (DV), 3.0 mm. On day 4 post-tumor implantation, mice were injected with 2.5×10⁶ TU of pLXIX-GAL4-IM and pAC3-GAL4BS-RLI. Bioluminescent imaging was then monitored biweekly. Mouse survival was also recorded; endpoint was considered weight loss >15% or the development of neurological symptoms.

### Analysis of Viral Spread

Mice were sacrificed at day 4,15, and 18 post-tumor injection time points. Day 15 and 18 time points were combined during analysis. Brain tumors were minced and placed into collagenase type IV (Thermo Fisher Scientific #17104019) and Deoxyribonuclease I (Worthington Biochemical Corporation) solutions for processing while shaking at 37 C. Tumors were then filtered through 70 um filters, and red blood cells were lysed using Ammonium-Chloride-Potassium (ACK) lysing buffer (Lonza). Flow cytometric analysis was then performed. Acquisition was conducted on an Attune NxT Flow Cytometer (Thermo Fisher Scientific). Analysis of flow cytometry results was performed using FlowJo software.

### Analysis of Immune Alterations

Mice were sacrificed at endpoints or day 14 post-tumor injection time points. The following tissues were collected: spleen, bone marrow, blood, and brain tumor. Brain tumors were minced and placed into collagenase type IV (Thermo Fisher Scientific #17104019) and Deoxyribonuclease I (Worthington Biochemical Corporation) solutions for processing while shaking at 37 C. Tumors were then filtered through 70 um filters, and red blood cells were lysed using Ammonium-Chloride-Potassium (ACK) lysing buffer (Lonza). Spleens were smashed through a 40 um filter, then subjected to ACK lysis. Bone marrow was filtered through a 40 um filter then similarly subjected to ACK lysis. Flow cytometric analysis and staining were then performed. In brief, cells were first exposed to mouse Fc block in PBS with 2% bovine serum albumin. Following Fc Block, cells were washed then stained with Zombie Aqua fixable viability dye (BioLegend #423101) in PBS. Cells were then washed again then stained for surface markers in PBS with 2% bovine serum albumin. Following surface marker staining, cells were stained for intracellular markers using the eBioscience™ Foxp3/Transcription Factor Staining Buffer Set (Thermo Fisher Scientific #00-5523-00). Acquisition was conducted on an Attune NxT Flow Cytometer (Thermo Fisher Scientific). Analysis of flow cytometry results was performed using FlowJo software.

### Results

### Minimal and Maximal Insertions of GAL4 Binding Sites Differentially Modify Replicating Retrovirus Replication and Viral Transgene Expression

As described above, insertion/deletions in the U3 of the 3′ MMLV LTR to differentially modify viral replication and expression were made, providing two replicating viruses (RRVs) with varying amounts of dependence on GAL4/VP16 expression. When applied to SB28 tumor cells, a strawberry transgene carrying RRV with a minimal deletion (pAC-Minimal-Strawberry) demonstrated robust expression, but no viral replication over nine days in culture (FIG. 9A). An RRV with the maximal deletion (pAC-Maximal-Strawberry) demonstrated no expression at all in a similarly performed experiment. Stable expression of GAL4/VP16 in the SB28 tumor cells permitted replication of the pAC-Minimal-Strawberry virus (FIG. 9B).

In Vitro Addition of a GAL4VP16 Carrying Defective Retrovirus Permits Expression and Replication of Replicating Retroviruses with Minimal and Maximal Insertions of GAL4 Binding Sites

After confirming the lack of replication in pAC-Minimal-Strawberry and pAC-Maximal-Strawberry without concurrent GAL4/VP16 expression, we then performed an experiment to assess the ability for GAL4 to permit viral replication. First, we added pAC-Minimal-Strawberry and pLXIX-GAL4-EMD to SB28 cells at an MOI of 0.3 per virus. We then monitored viral spread via flow cytometry for EMD and Strawberry at regularly spaced time points. In contrast to pAC-Minimal-Strawberry alone, the addition of pLXIX-GAL4-EMD and pAC-Minimal-Strawberry together to SB28 glioblastoma tumor cells allowed for robust viral replication and spreading of both viruses (FIG. 10A. In addition, co-infected cells demonstrated higher expression of Strawberry expression, highlighting the synergistic nature of the binary replication system (FIG. 10B). A similar experiment was then conducted with the pAC-Maximal-Strawberry. Similarly, the addition of pLXIX-GAL4-EMD and pAC-Maximal-Strawberry together to SB28 glioblastoma tumor cells allowed for robust viral replication and spreading of both viruses to an extremely high percentage of double positive cells (FIG. 10)

### In Vivo Assessments of the Minimal and Maximal Binary Systems Demonstrate Robust Co-Infection and Replication

Following the success of in vitro experiments for both the Minimal and Maximal Binary systems, we then sought to test the two systems in an intracranial mouse tumor. In one set of experiments, the systems were assessed via implantation of pre-mixed tumors (4% co-infected cells and 96% uninfected tumor). Pre-mix experiments demonstrated robust replication and expansions of a co-infected tumor cell population (FIG. 11A-11D). Similar in vivo experiments performed using injection of the viruses at low viral titers, to challenge the system, also demonstrated excellent replication and a substantial co-infected tumor cell population (FIG. 12A-C). No fluorescent protein expression was detected 4 days after viral injection, further demonstrating the robust spread and replicative capacity of the system.

### Binary-IM Transduction Leads to the Expression of IL-7, FLT3L, and 4-1BBL In Vitro

100% Binary-IM (pLXIX-GAL4-IM and pAC3-GAL4BS-RLI) infected SB28 in a T75 plate in 10 mL of media efficiently secrete IL-7, RLI, and FLT3L at a concentration of 75 ng/mL or 0.09 pg/cell/48 hours (FIG. 22). In addition, all infected cells expressed 4-1BBL.

### Binary-IM Treatment Leads to Decreased Tumor Growth and Increased Survival in Two Poorly Immunogenic Murine Models of Glioblastoma

Binary-IM (pLXIX-GAL4-IM and pAC3-GAL4BS-RLI) treatment leads to reduced tumor growth in the SB28 and Tu2449 murine GBM models as measured by bioluminescence and survival (i.e., BLI signal)(FIGS. 23A and 23B). Binary-IM treatment leads to complete eradication of implanted cells in treated SB28 and Tu2449 (FIGS. 23A and 23B).

### Binary-IM Treatment Increases Tumor Infiltration of Lymphocytes and Dendritic Cells in SB28 Treated Mice

Subsequent flow cytometric analysis of Binary-IM SB28 treated mice revealed significant alterations in the tumor immune microenvironment system relative to control mice. Binary-IM treatment increased tumor infiltration of lymphocytes at a 14 day time point, including CD3+ immune cell infiltration (3.0% vs. 20.0%, p=0.00l) and CD8 T cell infiltration (0.8% vs. 8.0%, p=0.01) (Binary-IM (i.e., treatment with pLXIX-GAL4-IM and pAC3-GAL4BS-RLI) is the middle column of each set of three columns (left: PBS control, middle:

Binary-IM; right: empty RRV) for each type of cell). There was no significant difference in Treg (CD3+, CD4+, CD25+, FOXP3+) infiltration. (FIG. 24). Plasmacytoid dendritic cell (pDC) infiltration was similarly increased at the day 14 time point (3.0% vs. 15.0%, p=0.001).

Exemplary 3′LTR and/or 5′ LTR sequences
Wildtype (unmodified) MMLV LTR sequence
(SEQ ID NO: 1)
AATGAAAGACCCCACCTGTAGGTTTGGCAAGCTAGCTTAAGTAACGCCA
TTTTGCAAGGCATGGAAAAATACATAACTGAGAATAGAGAAGTTCAGAT
CAAGGTCAGGAACAGATGGAACAGCTGAATATGGGCCAAACAGGATATC
TGTGGTAAGCAGTTCCTGCCCCGGCTCAGGGCCAAGAACAGATGGAACA
GCTGAATATGGGCCAAACAGGATATCTGTGGTAAGCAGTTCCTGCCCCG
GCTCAGGGCCAAGAACAGATGGTCCCCAGATGCGGTCCAGCCCTCAGCA
GTTTCTAGAGAACCATCAGATGTTTCCAGGGTGCCCCAAGGACCTGAAA
TGACCCTGTGCCTTATTTGAACTAACCAATCAGTTCGCTTCTCGCTTCT
GTTCGCGCGCTTCTGCTCCCCGAGCTCAATAAAAGAGCCCACAACCCCT
CACTCGGGGCGCCAGTCCTCCGATTGACTGAGTCGCCCGGGTACCCGTG
TATCCAATAAACCCTCTTGCAGTTGCATCCGACTTGTGGTCTCGCTGTT
CCTTGGGAGGGTCTCCTCTGAGTGATTGACTACCCGTCAGCGGGGGTCT
TTCATT
Minimal deletion MMLV LTR sequence
(SEQ ID NO: 2)
AATGAAAGACCCCACCTGTAGGTTTGGCAAGATGGATCCTCGATAGGTA
CCGAGTTTCTAGACGGAGTACTGTCCTCCGAGCGGAGTACTGTCCTCCG
ACTCGAGCGGAGTACTGTCCTCCGATCGGAGTACTGTCCTCCGCGAATT
CCGGAGTACTGTCCTCCGAAGACGCTAGACGGAGTACTGTCCTCCGAGC
GGAGTACTGTCCTCCGACTCGAGCGGAGTACTGTCCTCCGAAGACGCTA
GCGGGGGGCAGCAGTTTCTAGAGAACCATCAGATGTTTCCAGGGTGCCC
CAAGGACCTGAAATGACCCTGTGCCTTATTTGAACTAACCAATCAGTTC
GCTTCTCGCTTCTGTTCGCGCGCTTCTGCTCCCCGAGCTCAATAAAAGA
GCCCACAACCCCTCACTCGGGGCGCCAGTCCTCCGATTGACTGAGTCGC
CCGGGTACCCGTGTATCCAATAAACCCTCTTGCAGTTGCATCCGACTTG
TGGTCTCGCTGTTCCTTGGGAGGGTCTCCTCTGAGTGATTGACTACCCG
TCAGCGGGGGTCTTTCATT
Maximal deletion MMLV LTR sequence
(SEQ ID NO: 3)
AATGAAAGACCCCACCTGTAGGTTTGGCAAGATGGATCCTCGATAGGTA
CCGAGTTTCTAGACGGAGTACTGTCCTCCGAGCGGAGTACTGTCCTCCG
ACTCGAGCGGAGTACTGTCCTCCGATCGGAGTACTGTCCTCCGCGAATT
CCGGAGTACTGTCCTCCGAAGACGCTAGACGGAGTACTGTCCTCCGAGC
GGAGTACTGTCCTCCGACTCGAGCGGAGTACTGTCCTCCGAAGACGCTA
GCGGGGGGCCTCAATAAAAGAGCCCACAACCCCTCACTCGGGGCGCCAG
TCCTCCGATTGACTGAGTCGCCCGGGTACCCGTGTATCCAATAAACCCT
CTTGCAGTTGCATCCGACTTGTGGTCTCGCTGTTCCTTGGGAGGGTCTC
CTCTGAGTGATTGACTACCCGTCAGCGGGGGTCTTTCATT

What is claimed is:

1. A recombinant retrovirus system comprising:
(a) a first retrovirus (i) encoding a first regulatory element operably linked to a nucleic acid encoding a first activator; and (ii) lacking a coding sequence for at least one viral protein required for replication such that the first retrovirus is a replication-deficient retrovirus (RDV); and
(b) a second retrovirus comprising a nucleic acid comprising a first polynucleotide encoding the viral protein or proteins necessary for viral replication that are lacking in the first retrovirus, wherein the first polynucleotide is only expressed when the first activator activates expression of the first polynucleotide and/or its encoded viral protein(s).

2. The recombinant retrovirus system of claim 1, wherein the activator activates expression by increasing transcription or translation of the first polynucleotide.

3. The recombinant retrovirus system of claim 1 or 2, wherein the second retrovirus comprises a second regulatory element operably linked to the first polynucleotide encoding a viral protein necessary for viral replication, and wherein the first activator activates transcription of the first polynucleotide by binding to the second regulatory element.

4. The recombinant retrovirus system of any of claims 1-3, wherein the activator binds to the second regulatory element to activate transcription, wherein the second regulatory element is a promoter, an enhancer or a repressor binding sequence.

5. The recombinant retrovirus system of claim 1, wherein the first activator is a de-repressor; and the first polynucleotide sequence encoded by the second retrovirus is only expressed when the de-repressor activates expression by de-repressing expression of the first polynucleotide and/or viral protein(s).

6. The recombinant retrovirus system of claim 5, wherein de-repression occurs at the transcriptional level or the translational level.

7. The recombinant retrovirus system of any one of claims 1-6, wherein the first regulatory element and/or the second regulatory element is selected from the group consisting of a promoter, an enhancer, a promoter/enhancer combination, an internal ribosome entry site, an epigenetic regulator and a translation regulator.

8. The recombinant retrovirus system of claim 7, wherein the promoter is a constitutive or an inducible promoter.

9. The recombinant retrovirus system of any one of claims 1-8, wherein the second retrovirus is a replicating retrovirus (RRV) encoding all viral proteins necessary for viral replication.

10. The recombinant retrovirus system of any one of claims 1-9, wherein the first and/or second retrovirus further comprises a heterologous expression cassette comprising a payload promoter operably linked to a payload polynucleotide sequence.

11. The recombinant retrovirus system of any one of claims 1-10, wherein the viral protein required for replication is selected from the group consisting of gag, env, pol, rev and tat.

12. The recombinant retrovirus system of any one of claims 1-11, wherein the retroviruses are selected from the group consisting of lentivirus, murine leukemia virus (MLV), Moloney murine leukemia virus (MoMLV), foamy virus.

13. The recombinant retrovirus system of any one of claims 10-12, wherein the payload polynucleotide encodes a polypeptide selected from the group consisting of a therapeutic protein, a prodrug activator, a cytotoxic protein, and a reporter protein.

14. The recombinant retrovirus system of claim 13, wherein the prodrug activator is thymidine kinase, cytidine deaminase or purine nucleoside phosphorylase (PNP).

15. The recombinant retrovirus system of any one of claims 1-14, wherein the first activator is selected from the group consisting of HIV-I trans-activator protein (Tat), HIV-1 Rev, Gal4-VP16, GAL4FF, GAL4-VP64, and VP16-E2 and tetracycline transactivator protein.

16. The recombinant retrovirus system of claim 15, wherein the first activator is GAL4-VP16, and wherein the GAL4-VP16 binds to one or more GAL4 binding sites in the second retrovirus.

17. The recombinant retrovirus system of claim 1, wherein the second retrovirus is a replication-deficient retrovirus (RDV) and encodes a third regulatory element operably linked to a nucleic acid encoding a second activator; and the first retrovirus comprises a nucleic acid comprising a second polynucleotide encoding a viral protein necessary for viral replication, wherein the second polynucleotide is only expressed when the second activator activates expression of the second polynucleotide, and wherein the first and second retroviruses can only replicate when the first and second activators are expressed.

18. The recombinant retrovirus system of claim 17, wherein the first retrovirus comprises a fourth regulatory element operably linked to the second polynucleotide encoding a viral protein necessary for viral replication, and wherein the first activator activates transcription of the second polynucleotide by binding to the fourth regulatory element.

19. The recombinant retrovirus system of claim 17 or 18, wherein the third regulatory element or the fourth regulatory element is selected from the group consisting of a promoter, an enhancer, a promoter/enhancer combination, an internal ribosome entry site, an epigenetic regulator and a translational regulator.

20. The recombinant retrovirus system of claim 19, wherein the promoter is a constitutive or an inducible promoter.

21. The recombinant retrovirus system of any of claims 18-20, wherein the first retrovirus or second retrovirus, or both, comprise a heterologous expression cassette comprising a payload promoter operably linked to a payload polynucleotide.

22. The recombinant retrovirus system of claim 21, wherein the payload polynucleotide encodes a polypeptide selected from the group consisting of a therapeutic protein, a prodrug activator, a cytotoxic protein, and a reporter protein.

23. The recombinant retrovirus system of any one of claims 17-22, wherein the viral protein required for replication is selected from the group consisting of gag, env, pol, rev and tat.

24. The recombinant retrovirus system of claims 17-23, wherein the retroviruses are selected from the group consisting of lentivirus, murine leukemia virus (MLV), Moloney murine leukemia virus (MoMLV), and foamy virus.

25. The recombinant retrovirus system of claims 17-24, wherein the payload polynucleotide encodes a polypeptide selected from the group consisting of a therapeutic protein, a prodrug activator, a cytotoxic protein, and a reporter protein.

26. The recombinant retrovirus system of claim 25, wherein the prodrug activator is thymidine kinase, cytidine deaminase or purine nucleoside phosphorylase (PNP).

27. The recombinant retrovirus system of any one of claims 17-26, wherein the third or fourth regulatory element is selected from the group consisting of a constitutive promoter, an inducible promoter, and a tissue-specific promoter.

28. The recombinant retrovirus system of any one of claims 17-27, wherein the activator is selected from the group consisting of HIV-1 trans-activator protein (Tat), HIV-1 Rev, Gal4-VP16, and VP16-E2 and tetracycline transactivator protein.

29. A method for making a recombinant retrovirus system comprising:
(a) transfecting a first suitable host cell with the first retroviral vector of any of claims 1-28;
(b) transfecting a second suitable host cell with the second retroviral vector of any of claims 1-28; and
(c) recovering the first and second retroviruses.

30. A method for transfecting a target cell with a replicating retrovirus system comprising contacting the target cell with the first retrovirus and second retrovirus of any one of claims 1-29.

31. The method of claim 30, wherein the cell is a mammalian cell.

32. The method of any of claims 29-31, wherein the cell is contacted in vitro, ex vivo or in vivo.

33. A pharmaceutical composition comprising:
(a) the first retrovirus and/or the second retrovirus of the system of any of claims 1-28:
(b) and a pharmaceutical carrier.

34. A method of treating a disease in a subject in need thereof comprising administering the system of any one of claims 1-28, or the pharmaceutical composition of claim 33, to the subject.

35. The method of claim 34, wherein the disease is a cell proliferative disorder.

36. The method of claim 35, wherein the cell proliferative disorder is selected from the group consisting of lung cancer, breast cancer, ovarian cancer, uterine cancer, prostate cancer, testicular cancer, kidney cancer, urinary tract cancer, oral cancer, head and neck cancer, esophageal cancer, gastric cancer, pancreatic cancer, colorectal cancer, skin cancer, melanoma, sarcoma, lymphoma, leukemia, and brain cancer including glioblastoma, anaplastic astrocytoma, oligodendroglioma, medulloblastoma.

37. The method of claim 36, wherein the cancer is glioblastoma.

38. The method of any one of claims 34-37, wherein the first or second polynucleotide encodes a prodrug activator, and wherein the method further comprises administering a prodrug to the subject, such that when the prodrug activator is expressed, the prodrug activator converts the prodrug into a toxic drug.

39. The method of any one of claims 34-38, wherein the first and/or second retroviral vector is administered to the subject as a plasmid or as an infectious retroviral particle.

40. The method of any one of claims 34-39, wherein the subject is a mammal.

41. The method of claim 40, wherein the subject is a human.

42. The method of any of claims 34-41, wherein administration is systemic, topical or local administration.

43. The method of any one of claims 34-42, wherein the first and second retrovirus of the system are administered simultaneously or sequentially to the subject.


SEQUENCE LISTING (13 sequences)

SEQ ID NO: 1 - DNA, 594 bp, synthetic construct
  source 1..594: mol_type = other DNA
aatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatt

SEQ ID NO: 2 - DNA, 558 bp, Synthetic construct
  source 1..558: mol_type = other DNA
aatgaaagaccccacctgtaggtttggcaagatggatcctcgataggtaccgagtttctagacggagtactgtcctccgagcggagtactgtcctccgactcgagcggagtactgtcctccgatcggagtactgtcctccgcgaattccggagtactgtcctccgaagacgctagacggagtactgtcctccgagcggagtactgtcctccgactcgagcggagtactgtcctccgaagacgctagcggggggcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatt

SEQ ID NO: 3 - DNA, 432 bp, synthetic construct
  source 1..432: mol_type = other DNA
aatgaaagaccccacctgtaggtttggcaagatggatcctcgataggtaccgagtttctagacggagtactgtcctccgagcggagtactgtcctccgactcgagcggagtactgtcctccgatcggagtactgtcctccgcgaattccggagtactgtcctccgaagacgctagacggagtactgtcctccgagcggagtactgtcctccgactcgagcggagtactgtcctccgaagacgctagcggggggcctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatt

SEQ ID NO: 4 - DNA, 22 bp, synthetic construct
  source 1..22: mol_type = other DNA
cggagtactgtcctccgagcgg

SEQ ID NO: 5 - DNA, 60 bp, synthetic construct
  source 1..60: mol_type = other DNA
tttcttggctttatatatcttgtggaaaggacgaaacaccgagtagcgcgagcacagcta

SEQ ID NO: 6 - DNA, 60 bp, synthetic construct
  source 1..60: mol_type = other DNA
gactagccttattttaacttgctatttctagctctaaaactagctgtgctcgcgctactc

SEQ ID NO: 7 - DNA, 527 bp, synthetic construct
  source 1..527: mol_type = other DNA
atgaagcttctctctagcattgaacaggcatgtgatatctgccgactcaagaaactaaaatgctccaaggagaagccaaagtgtgccaagtgcttgaaaaataactgggagtgtcgctacagtcccaagaccaagcgttctccgctgactcgggctcatctcaccgaagtcgagagccgcctggaacgtttagagcaattgttcctcctcatcttcccccgggaagatcttgacatgatccttaagatggactccttgcaggatatcaaggccctcttgaccgggctattcgttcaagacaatgtcaacaaggacgcagtcacagaccgccttgcgagcgtggagactgatatgccactgacgctaaggcagcacagaatttcagccacaagtagttcagaagaatcttccaataaagggcagcggcaattgactgtctccccagagttccctgctgacgccttggatgatttcgacctcgacatgcttccagcagatgctcttgatgactttgatttggatatgct

SEQ ID NO: 8 - DNA, 261 bp, synthetic construct
  source 1..261: mol_type = other DNA
atggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcaacccacctcccaaccccgaggggacccgacaggcccgaaggaatag

SEQ ID NO: 9 - DNA, 351 bp, synthetic construct
  source 1..351: mol_type = other DNA
atggcaggaagaagcggagacagcgacgaagagctcatcagaacagtcagactcatcaagcttctctatcaaagcaacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatag

SEQ ID NO: 10 - DNA, 747 bp, synthetic construct
  source 1..747: mol_type = other DNA
atgtctagactggacaagagcaaagtcataaacggagctctggaattactcaatggtgtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaa

SEQ ID NO: 11 - DNA, 57 bp, synthetic construct
  source 1..57: mol_type = other DNA
ggtctctctggttagaccagatctgagcctgggagctctctggctaactagggaacc

SEQ ID NO: 12 - DNA, 60 bp, synthetic construct
  source 1..60: mol_type = other DNA
tttcttggctttatatatcttgtggaaaggacgaaacaccggaaaagaaaggtaattatg

SEQ ID NO: 13 - DNA, 60 bp, synthetic construct
  source 1..60: mol_type = other DNA
gactagccttattttaacttgctatttctagctctaaaaccataattacctttcttttcc